SEQUENCE LISTING (54 sequences)

SEQ ID NO: 1 - DNA, 300 bp, Rattus rattus
attaatgtactcactcttggcaaaacaaaactggctcaggagaaaaattgtgtatttgcaattccgacattatgtgtaacacaatctctttcttcttttagtgacgtctccaaaattaatgaaggaattggtgacaaaattggaatgttctttcaggcaatggcaacattttttggtggttttataataggatttactcgcggctggaagctaactcttgtgattttggccatcagccctgttcttggactgtcagctggtatttgggcaaaggtaggtgaagcccgtgagtccagattt

SEQ ID NO: 2 - DNA, 200 bp, Rattus rattus
gtgacgtctccaaaattaatgaaggaattggtgacaaaattggaatgttctttcaggcaatggcaacattttttggtggttttataataggatttactcgcggctggaagctaactcttgtgattttggccatcagccctgttcttggactgtcagctggtatttgggcaaaggtaggtgaagcccgtgagtccagattt

SEQ ID NO: 3 - DNA, 400 bp, Rattus rattus
ggtaaaaggaacaattgattgacagcacgcttgcttggtagagtctgcctgaagtatacagttcacactagattatgtgcgtggcagggctcttctgtttgctgagtgacttgtcactggctttttcttagaacctgggcccttctgtgctggctggggtggctgttatgatcctcatggtgcccttcaatgctgtgatggccatgaagaccaagacttaccaggtaggatgtccaactccatgagacttcattctgagccctggcctgggtctttccaggtgagggtcgcccagtttcctgatgcttgggcctaggatagacagcctaatctattataggtgtcataatacagggatactgcttccctcagtggccttcaaatgtctgcaagtttgcca

SEQ ID NO: 4 - DNA, 200 bp, Rattus rattus
gctgagtgacttgtcactggctttttcttagaacctgggcccttctgtgctggctggggtggctgttatgatcctcatggtgcccttcaatgctgtgatggccatgaagaccaagacttaccaggtaggatgtccaactccatgagacttcattctgagccctggcctgggtctttccaggtgagggtcgcccagtttcc

SEQ ID NO: 5 - DNA, 900 bp, Rattus rattus
atgcacatactttaaaaatcaactcattaaatttaaatatctaacaacaaggatatcctaactatctatctgataagaatcataatttgaaagcagcaattactgtgagttagctatggaaacatctacggtttctcctggtaacaaagacaccatgttatgctcatattaggagaaaaggctggatctcagttatggattgttacaaataaaggtgaagctagttttccatccacattctccagccctgaattggataggcccgatctagacatagactgtgaagacagtggtgaggtcactccatttcctaagggcatcatggagctccctcgaggacctgtcagagtgtagagaggtggccggtacctgatggagaaggagccataggaggacttttgattcaatagagcctctctccgtctcttctccatcctgtcttctagcatattctcttgtctctgtgctctccccccacgcttctgtcaaataaaggaccaggagatgtagacagtggaacagtagagaagtccaatgcttcatcctctgcctcagatctggaggataggttctgacagaagcagactgacgtagcccagagagactcagcctgtggactgttctcattccaggactgttctgaaaggttacaagcatccactgacactagaagatgtctgggatatcgatgaagggtttaaaacaaggtcagtcaccagcaagtttgaggcggccatgacaaaggacctgcagaaagccaggcaggcttttcagaggcggctgcagaagtcccagcggaaacctgaggccacactacacggactgaacaagaagcagagtcagagccaagacgttctcgtcctggtaactttaactcaagtgtccgtgtgaatgcactgt

SEQ ID NO: 6 - DNA, 900 bp, Rattus rattus
ccaggcccacgtgtggcacacaggcatatgtgcaggcaaaatacccatatacatcaaaaaaaatttttaagaaaaaatagaataattgagcctaaataagtggggagttagtagctatgaatttgaaataggcgttaggccagtttgagagaactgtaaagttgagaccagagctaaagtgtgctgtcctgtctatgttctcttgagaatgacagaagagaattagaatctctccacttagtaactgaagagacaatgatgacgtgcagatcttgaagtcagagagcacaagaacagttaatgttttcccttccttccttccttccttccttccttccttccttccttccttccttctttctttctttctggacttggatttagatctcagacagaagactagtgagcaccagagcatatcagtagtcctccgtggctcataatttatgatatacaccaatgcatgagtttctcaagtgtccaaaactcctctggaattattggagtatttgttaagcatatgagaaaaggatgtgttagtgaagggagagtgagaatggggagaacttggcgcgttctgtcttcccaagccttccctgcctcatctgaatacaccttctctattccaagaccatcagggtcgtgtgtccttgtgttgccttctcttgcaggttaccactgtgagccctacaacaaccctgcagacttcttccttgacgtcatcaacggagattcttcggctgtaatgttaaacagaggggaacaagaccatgaaggtatatgagtcttgtagattcatacgttggtgtccaattgtttgattgaagcccagtagtgtgtcttattaaagcatgcttttgtaggccagccaggactatgtagtgagatcctaccttaaaa

SEQ ID NO: 7 - DNA, 800 bp, Rattus rattus
tcttgagaatgacagaagagaattagaatctctccacttagtaactgaagagacaatgatgacgtgcagatcttgaagtcagagagcacaagaacagttaatgttttcccttccttccttccttccttccttccttccttccttccttccttccttctttctttctttctggacttggatttagatctcagacagaagactagtgagcaccagagcatatcagtagtcctccgtggctcataatttatgatatacaccaatgcatgagtttctcaagtgtccaaaactcctctggaattattggagtatttgttaagcatatgagaaaaggatgtgttagtgaagggagagtgagaatggggagaacttggcgcgttctgtcttcccaagccttccctgcctcatctgaatacaccttctctattccaagaccatcagggtcgtgtgtccttgtgttgccttctcttgcaggttaccactgtgagccctacaacaaccctgcagacttcttccttgacgtcatcaacggagattcttcggctgtaatgttaaacagaggggaacaagaccatgaaggtatatgagtcttgtagattcatacgttggtgtccaattgtttgattgaagcccagtagtgtgtcttattaaagcatgcttttgtaggccagccaggactatgtagtgagatcctaccttaaaacaaaaatcgtgggttggggattttgctcagtggtagagcgcttgcctagaaagcgcaaggccctgggttcagtccccagctccgaaaaaaaagaaccaaa

SEQ ID NO: 8 - DNA, 28 bp, Rattus rattus
acagggctgatggccaaaatcacaagag

SEQ ID NO: 9 - DNA, 28 bp, Rattus rattus
ttggactgtcagctggtatttgggcaaa

SEQ ID NO: 10 - DNA, 28 bp, Rattus rattus
aggaggggaagcagggttccgtggatga

SEQ ID NO: 11 - DNA, 28 bp, Rattus rattus
atgctggtgttcggatacatgacagata

SEQ ID NO: 12 - DNA, 28 bp, Rattus rattus
gaagggcccaggttctaagaaaaagcca

SEQ ID NO: 13 - DNA, 28 bp, Rattus rattus
tgctggctggggtggctgttatgatcct

SEQ ID NO: 14 - DNA, 28 bp, Rattus rattus
ttgctggtgactgaccttgttttaaacc

SEQ ID NO: 15 - DNA, 28 bp, Rattus rattus
ttgaggcggccatgacaaaggacctgca

SEQ ID NO: 16 - DNA, 28 bp, Rattus rattus
atgacgtcaaggaagaagtctgcagggt

SEQ ID NO: 17 - DNA, 28 bp, Rattus rattus
acggagattcttcggctgtaatgttaaa

SEQ ID NO: 18 - DNA, 29 bp, Rattus rattus
ctgtttcttgacaaaacaacactaggctc

SEQ ID NO: 19 - DNA, 25 bp, Rattus rattus
gggtcatgggaaagagtttaaaatc

SEQ ID NO: 20 - DNA, 20 bp, Rattus rattus
catgctgtgaagcagatacc

SEQ ID NO: 21 - DNA, 24 bp, Rattus rattus
ctgaaaactgaatgagacatttgc

SEQ ID NO: 22 - DNA, 22 bp, Rattus rattus
gccgataaaagagccatgtttg

SEQ ID NO: 23 - DNA, 21 bp, Rattus rattus
gataaggagaaaagctgcacc

SEQ ID NO: 24 - DNA, 33 bp, Rattus rattus
gccatcagccctgttcttggactgtcagctggt

SEQ ID NO: 25 - DNA, 33 bp, Rattus rattus
gccatcagctctcaaagaggactgtaagaagct

SEQ ID NO: 26 - DNA, 32 bp, Rattus rattus
gccaacagctctattttggactctccgctgct

SEQ ID NO: 27 - DNA, 33 bp, Rattus rattus
gccatcagctctataacatgactgtctactgat

SEQ ID NO: 28 - DNA, 33 bp, Rattus rattus
gtcaccaaccctctccatggaaagtcagctggt

SEQ ID NO: 29 - DNA, 33 bp, Rattus rattus
gacttcagccctgactgctgactggcaactggt

SEQ ID NO: 30 - DNA, 33 bp, Rattus rattus
gccagcagccctttccttgaagggtcagctagt

SEQ ID NO: 31 - DNA, 33 bp, Rattus rattus
gccatcagcccgctcatgagcctgtttgctggt

SEQ ID NO: 32 - DNA, 32 bp, Rattus rattus
gccagcagccctgcctgggcctggcagttagt

SEQ ID NO: 33 - DNA, 33 bp, Rattus rattus
gctgtcagccctcttattggattgtcatctgct

SEQ ID NO: 34 - DNA, 33 bp, Rattus rattus
gccctcagccctcgagatgctctgtcatcaggt

SEQ ID NO: 35 - DNA, 32 bp, Rattus rattus
gccatcagcccactgtgggactttgagtgggt

SEQ ID NO: 36 - DNA, 32 bp, Rattus rattus
cacctgagcccgcaactggactgtcagctggt

SEQ ID NO: 37 - DNA, 33 bp, Rattus rattus
cccatcaacactaacacaggactggcatctggt

SEQ ID NO: 38 - DNA, 32 bp, Rattus rattus
tccagcagctctgtctgggactgttagatggt

SEQ ID NO: 39 - DNA, 32 bp, Rattus rattus
cccaacagccctattagggacaggcacctggt

SEQ ID NO: 40 - DNA, 32 bp, Rattus rattus
gccatcaggcatggagaggacattcagctgga

SEQ ID NO: 41 - DNA, 32 bp, Rattus rattus
gccatcgcccctggcctggatggtctgctggt

SEQ ID NO: 42 - DNA, 33 bp, Rattus rattus
cccatcagcactgtggacggtcggtcatctggt

SEQ ID NO: 43 - DNA, 33 bp, Rattus rattus
gccaggagcctttcaagtggactgtcagttgct

SEQ ID NO: 44 - DNA, 33 bp, Rattus rattus
gccagcagctgtgactgtgggctatcagctggt

SEQ ID NO: 45 - protein, 28 aa, Artificial Sequence
  SYNTHESIZED
DRSHLSRTSGNLTRQSSDLSRRSDHLTQ

SEQ ID NO: 46 - protein, 35 aa, Artificial Sequence
  SYNTHESIZED
TSGHLSRQSSDLSRQSADRTKRSDVLSEQSGHLSR

SEQ ID NO: 47 - protein, 35 aa, Artificial Sequence
  SYNTHESIZED
TSGHLSRRSDNLSERNANRITRSDHLSERNDNRKR

SEQ ID NO: 48 - protein, 28 aa, Artificial Sequence
  SYNTHESIZED
RSDHLSENNSSRTRTSGHLSRQSSDLRR

SEQ ID NO: 49 - protein, 35 aa, Artificial Sequence
  SYNTHESIZED
TNGQLKETSSSLSRRSDNLSEASKTRKNRSDHLTQ

SEQ ID NO: 50 - protein, 35 aa, Artificial Sequence
  SYNTHESIZED
DRSALSRRSDALARRSDHLSRQSSDLRRRSDVLSE

SEQ ID NO: 51 - protein, 35 aa, Artificial Sequence
  SYNTHESIZED
TSDHLTEDRSNLSRDRSNLTRTSGHLSRQSSDLRR

SEQ ID NO: 52 - protein, 42 aa, Artificial Sequence
  SYNTHESIZED
RSDNLSVQNATRINRSDALSTDRSTRTKRSDDLSRRNDNRTK

SEQ ID NO: 53 - protein, 35 aa, Artificial Sequence
  SYNTHESIZED
QSGNLARQSGNLARRSDSLSTDNASRIRDRSNLTR

SEQ ID NO: 54 - protein, 35 aa, Artificial Sequence
  SYNTHESIZED
QSSDLSRRNDDRKKRREDLITTSSNLSRQSGHLSR

(19) United States
(12) Patent Application Publication
(10) Pub. No.: US 2012/0159654 A1
(43) Pub. Date: Jun. 21, 2012

(54) GENOME EDITING OF GENES INVOLVED IN ADME AND TOXICOLOGY IN ANIMALS

(75) Inventors: Edward Weinstein, St. Louis, MO (US); Xiaoxia Cui, St. Louis, MO (US); Phil Simmons, St. Louis, MO (US)

(73) Assignee: SIGMA-ALDRICH CO., St. Louis, MO (US)

(21) Appl. No.: 12/843,000

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. G01N 33/48; A01K 67/00; C12N 5/10

(52) U.S. Cl. 800/3; 435/325; 435/350; 435/351; 435/352; 435/353; 435/363; 800/13; 800/14; 800/15; 800/16; 800/17

(57) ABSTRACT

The present invention provides genetically modified animals and cells comprising edited chromosomal sequences involved in ADME and toxicology. In particular, the animals or cells are generated using a zinc finger nuclease-mediated editing process. The invention also provides zinc finger nucleases that target chromosomal sequence involved in ADME and toxicology and the nucleic acids encoding said zinc finger nucleases. Also provided are methods of assessing the effects of agents in genetically modified animals and cells comprising edited chromosomal sequences involved in ADME and toxicology.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence involved in ADME and toxicology. In particular, the invention relates to the use of a zinc finger nuclease-mediated process to edit chromosomal sequences involved in ADME and toxicology.

### BACKGROUND OF THE INVENTION

ADME is an acronym in pharmacokinetics and pharmacology for absorption, distribution, metabolism, and excretion (hereinafter “ADME”). ADME are criteria that affect the disposition of a pharmaceutical compound within an organism. Absorption includes how a compound reaches a tissue, namely, it must be taken into the bloodstream, often via mucous surfaces like the digestive tract, after being taken up by the targeted cells. Distribution affects how the compound is carried to its effector site, most commonly via the blood stream, before moving into tissues and organs. Distribution is defined as the reversible transfer of a drug between one compartment to another and factors affecting distribution include blood flow rates and the drug being to serum proteins to form a complex. Metabolism affects how the compounds break down when they enter the body. The majority of small-molecule drug metabolism is carried out in the liver by redox enzymes or cytochrome P450 enxyme. The body breaks the compound into metabolites as metabolism of the compound occurs, affecting how quickly or slowly a drug affects the organism. Excretion removes the metabolites from the body and when this does not occur, accumulation of foreign substances can adversely affect normal metabolism. Excretion of drugs by the kidney involves three main mechanisms: glomerular filtration of unbound drug, active secretion of drug by transporters, and filtrate 100-fold concentration in tubules for a favorable concentration gradient so that it may be reabsorbed by passive diffusion and passed out through the urine. Toxicity is also taken into account and either the potential or real toxicity of the drug is considered. The route of administration of a drug critically influences ADME and toxicity.

The vast majority of drugs (approximately 91%) fail to successfully complete the three phases of drug testing in humans. A majority of those drugs that fail, do so because of unforeseen toxicology in human patients, despite the fact that all of these drugs had been tested in animal models and were found to be safe. This is because toxicology testing is performed in animals, and animal proteins differ from the orthologous proteins in humans.

What is needed in the art are animals that are mutated for the genes involved in ADME and toxicology processes, including knockouts, multiple mutant lines (double knockouts, triple knockouts, etc.) and/or over-expression of alleles that either cause disease or are associated with disease in humans, as well as “humanized” animals that express or over-express human homologues of relevant genes in animals.

### SUMMARY OF THE INVENTION

One aspect of the present disclosure encompasses a genetically modified animal comprising at least one edited chromosomal sequence involved in ADME and toxicology.

A further aspect provides a non-human embryo comprising at least one RNA molecule encoding a zinc finger nuclease that recognizes a chromosomal sequence involved in ADME and toxicology, and, optionally, at least one donor polynucleotide comprising a sequence encoding an ortholog of a protein encoded by a chromosomal sequence involved in ADME and toxicology.

Another aspect provides a genetically modified cell comprising at least one edited chromosomal sequence involved in ADME and toxicology.

Yet another aspect encompasses a method for assessing the effect of an agent in an animal. The method comprises contacting a genetically modified animal comprising at least one edited chromosomal sequence involved in ADME and toxicology with the agent, and comparing results of a selected parameter to results obtained from contacting a wild-type animal with the same agent. The selected parameter is chosen from (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); and (g) efficacy of the agent or its metabolite(s).

Other aspects and features of the disclosure are described more thoroughly below.

### REFERENCE TO COLOR FIGURES

The application file contains at least one figure executed in color. Copies of this patent application publication with color figures will be provided by the Office upon request and payment of the necessary fee.

### BRIEF DESCRIPTION OF THE FIGURES

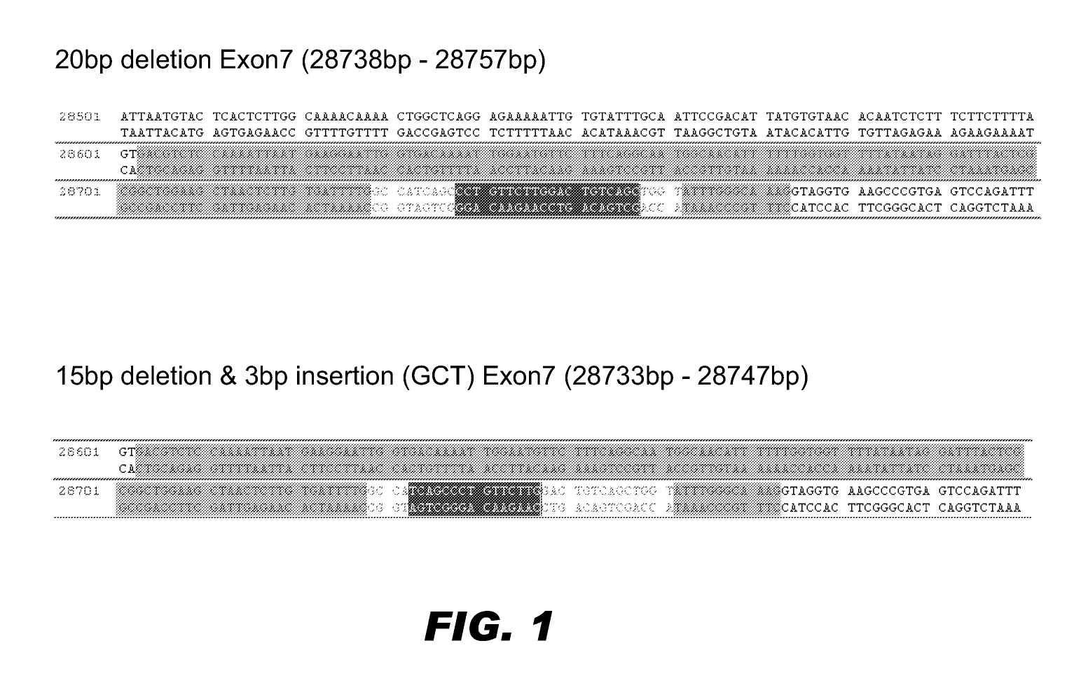

FIG. 1 presents the DNA sequences of edited Mdr1a loci in two animals. The upper sequence (SEQ ID NO:1) has a 20 bp deletion in exon 7, and the lower sequence (SEQ ID NO:2) has a 15 bp deletion and a 3 bp insertion (GCT) in exon 7. The exon sequence is shown in green; the target sequence is presented in yellow, and the deletions are shown in dark blue.

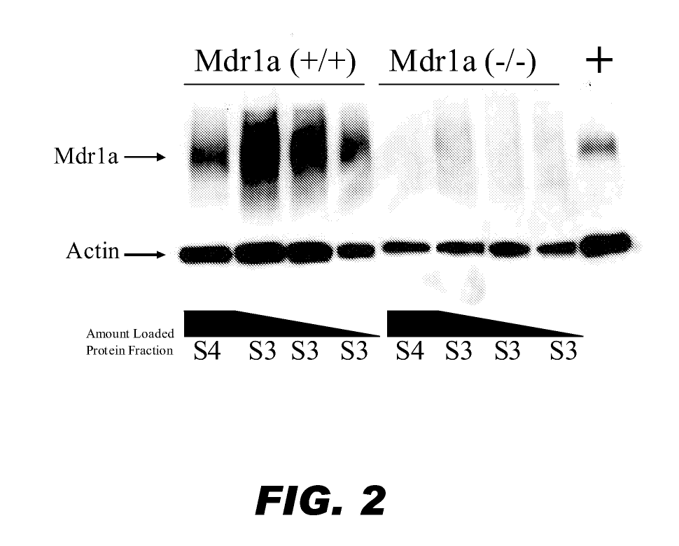

FIG. 2 illustrates knockout of the Mdr1a gene in rat. Presented is a Western blot of varying amounts of a colon lysate from an Mdr1a knockout rat and a control cell lysate. The relative locations Mdr1a protein and actin protein are indicated to the left of the image

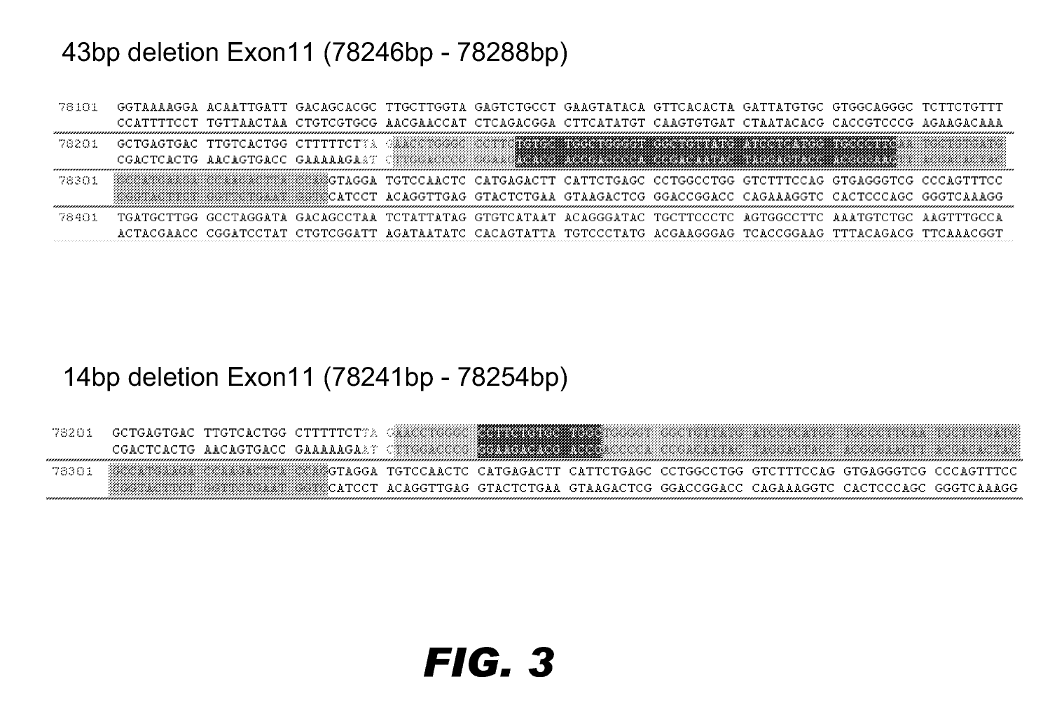

FIG. 3 presents the DNA sequences of edited Mrp1 loci in two animals. The upper sequence (SEQ ID NO:3) has a 43 bp deletion in exon 11, and the lower sequence (SEQ ID NO:4) has a 14 bp deletion in exon 11. The exon sequence is shown in green; the target sequence is presented in yellow, the deletions are shown in dark blue; and overlap between the target sequence and the exon is shown in grey.

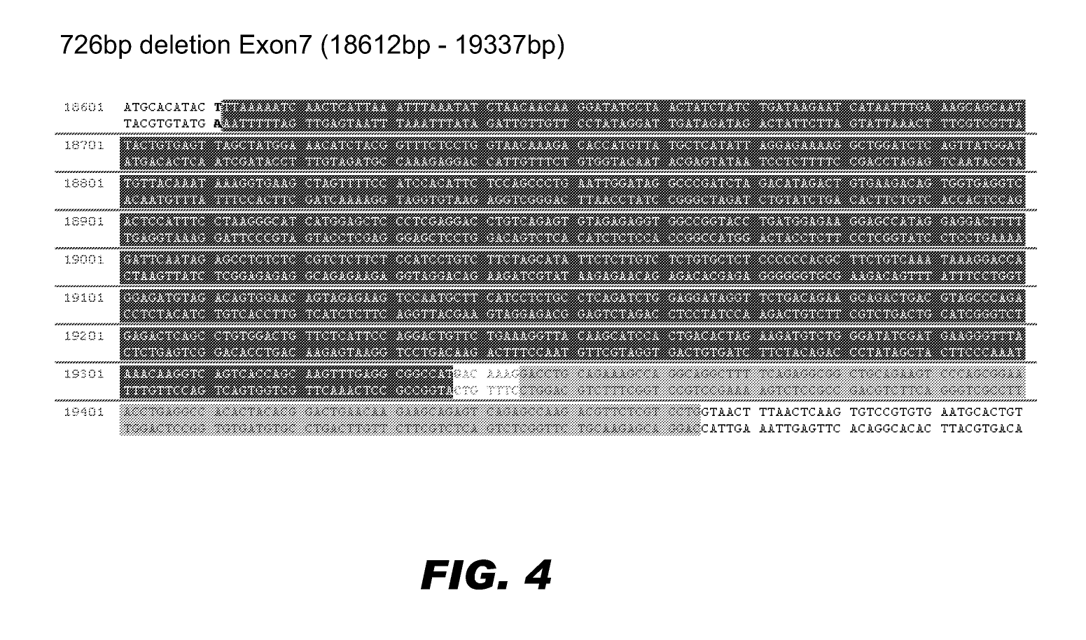

FIG. 4 shows the DNA sequence of an edited Mrp2 locus. The sequence (SEQ ID NO:5) has a 726 bp deletion in exon 7. The exon is shown in green; the target sequence is presented in yellow, and the deletion is shown in dark blue.

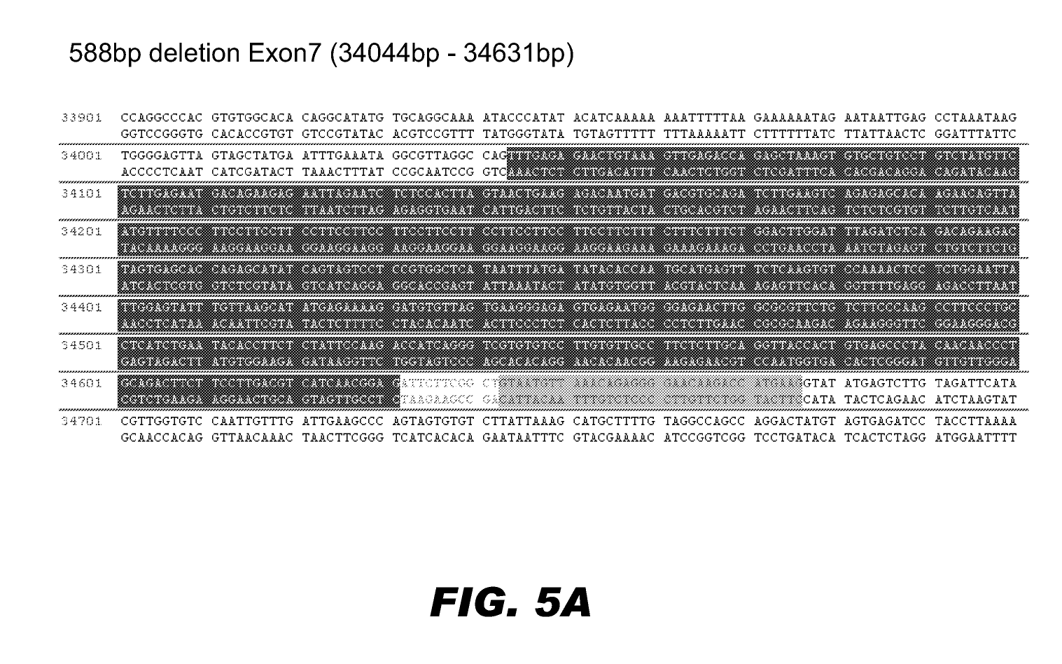

FIG. 5 presents the DNA sequences of edited BCRP loci in two animals. (A) Shows a region of the rat BCRP locus (SEQ ID NO: 6) comprising a 588 bp deletion in exon 7. (B) Presents a region of the rat BCRP locus (SEQ ID NO: 7) comprising a 696 bp deletion in exon 7. The exon sequence is shown in green; the target site is presented in yellow, and the deletions are shown in dark blue.

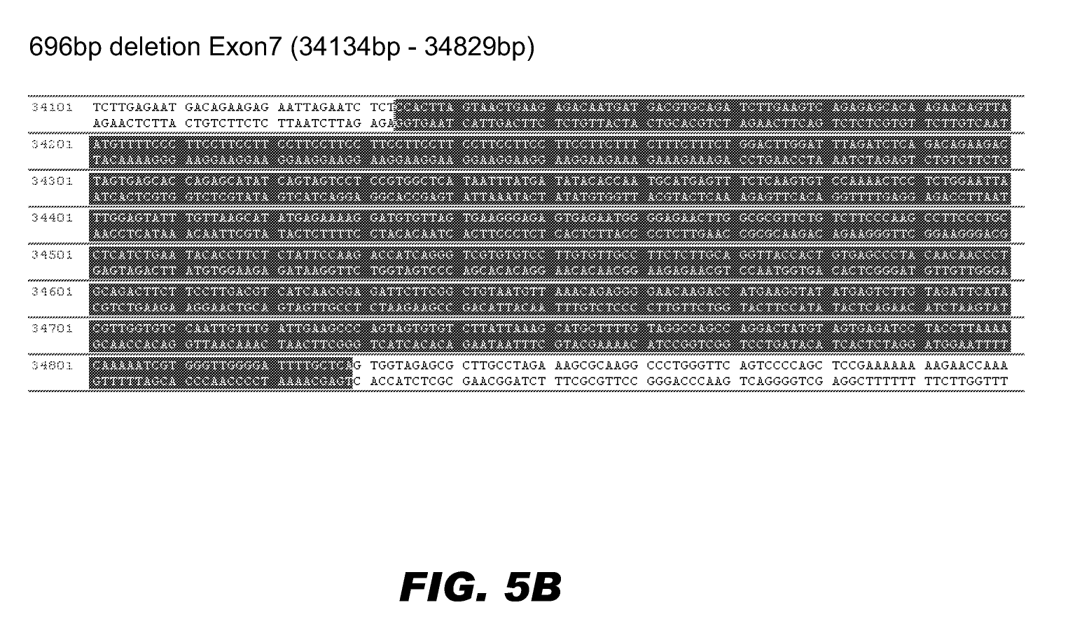

FIG. 6 presents target sites and ZFN validation of Mdr1a, and two additional genes, Jag1, and Notch3. (A) shows ZFN target sequences. The ZFN binding sites are underlined. (B) shows results of a mutation detection assay in NIH 3T3 cells to validate the ZFN mRNA activity. Each ZFN mRNA pair was cotransfected into NIH 3T3 cells. Transfected cells were harvested 24 h later. Genomic DNA was analyzed with the mutation detection assay to detect NHEJ products, indicative of ZFN activity. M, PCR marker; G (lanes 1, 3, and 5): GFP transfected control; Z (lanes 2, 4, and 6), ZFN transfected samples. Uncut and cleaved bands are marked with respective sizes in base pairs.

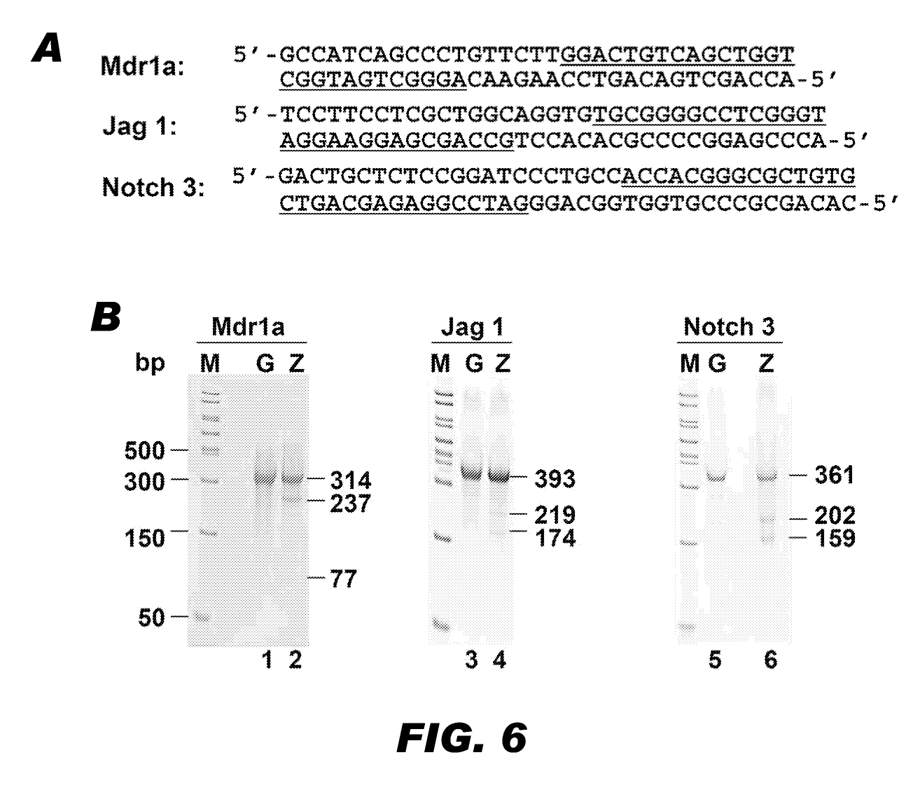

FIG. 7 presents identification of genetically engineered Mdr1a founders using a mutation detection assay. Uncut and cleaved bands are marked with respective sizes in base pairs. Cleaved bands indicate a mutation is present at the target site. M, PCR marker. 1-44, 44 pups born from injected eggs. The numbers of founders are underlined.

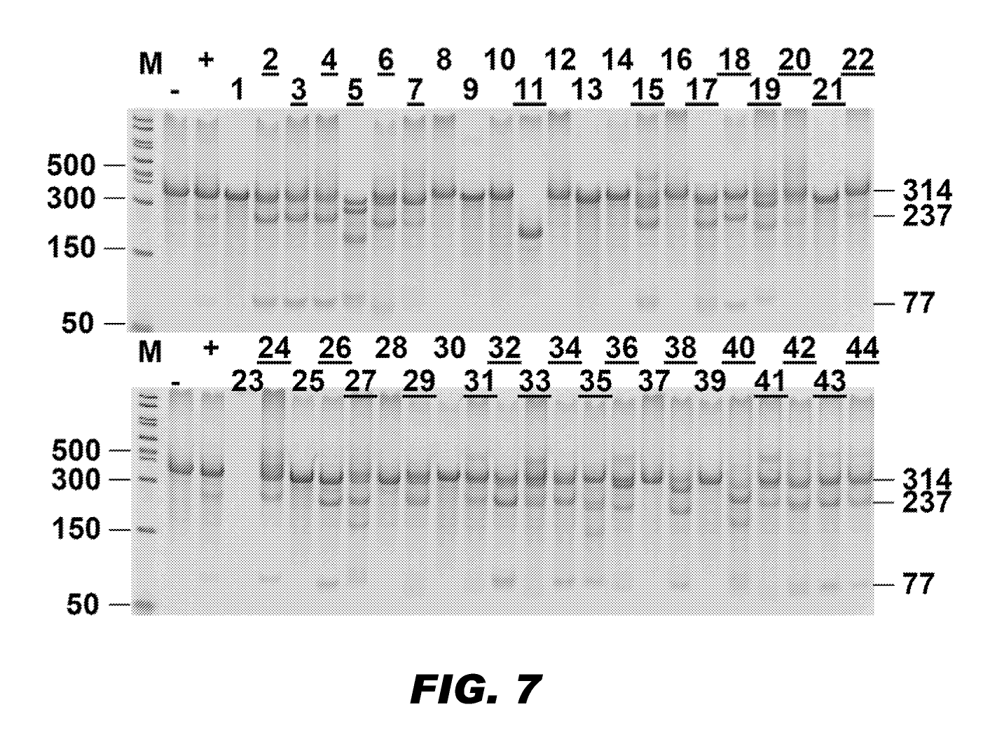

FIG. 8 presents amplification of large deletions in Mdr1a founders. PCR products were amplified using primers located 800 bp upstream and downstream of the ZFN target site. Bands significantly smaller than the 1.6 kb wild-type band indicate large deletions in the target locus. Four founders that were not identified in FIG. 7 are underlined.

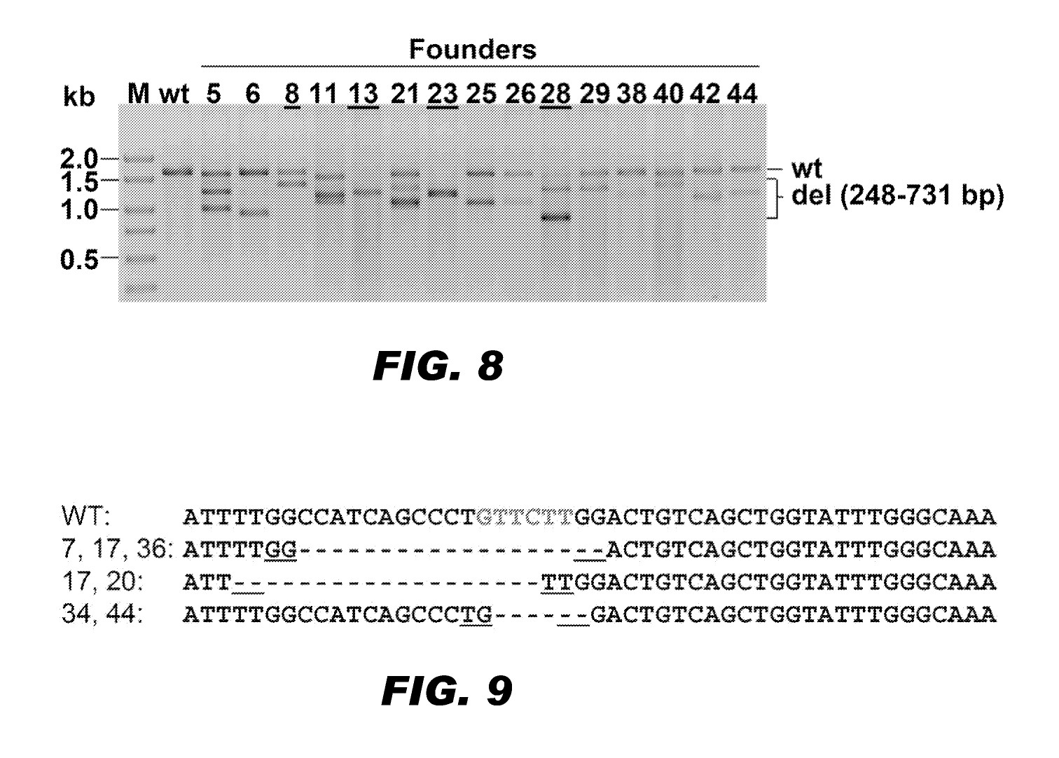

FIG. 9 presents the results of a mutation detection assay at the Mdr1b site in 44 Mdr1a ZFN injected pups. M, PCR marker; WT, toe DNA from FVB/N mice that were not injected with Mdr1a ZFNs; 3T3, NIH 3T3 cells transfected with Mdr1a ZFNs as a control.

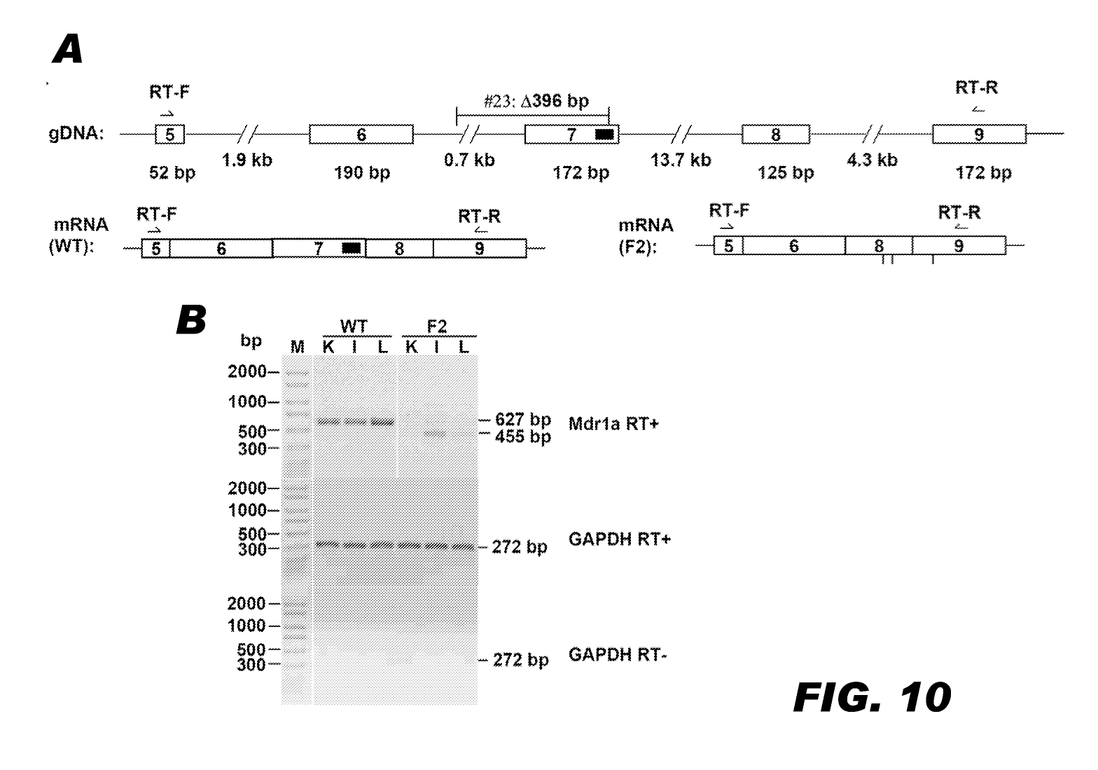

FIG. 10 presents detection Mdr1a expression by using RT-PCR in Mdr1a−/− mice. (A) is a schematic illustration of Mdr1a genomic and mRNA structures around the target site. Exons are represented by open rectangles with respective numbers. The size of each exon in base pair is labeled directly underneath. Intron sequences are represented by broken bars with size in base pairs underneath. The ZFN target site in exon 7 is marked with a solid rectangle. The position of the 396 bp deletion in founder #23 is labeled above intron 6 and exon 7. RT-F and RT-R are the primers used in RT-PCR, located in exons 5 and 9, respectively. In the RT reaction, 40 ng of total RNA was used as template. Normalization of the input RNA is confirmed by GAPDH amplification with or without RT.

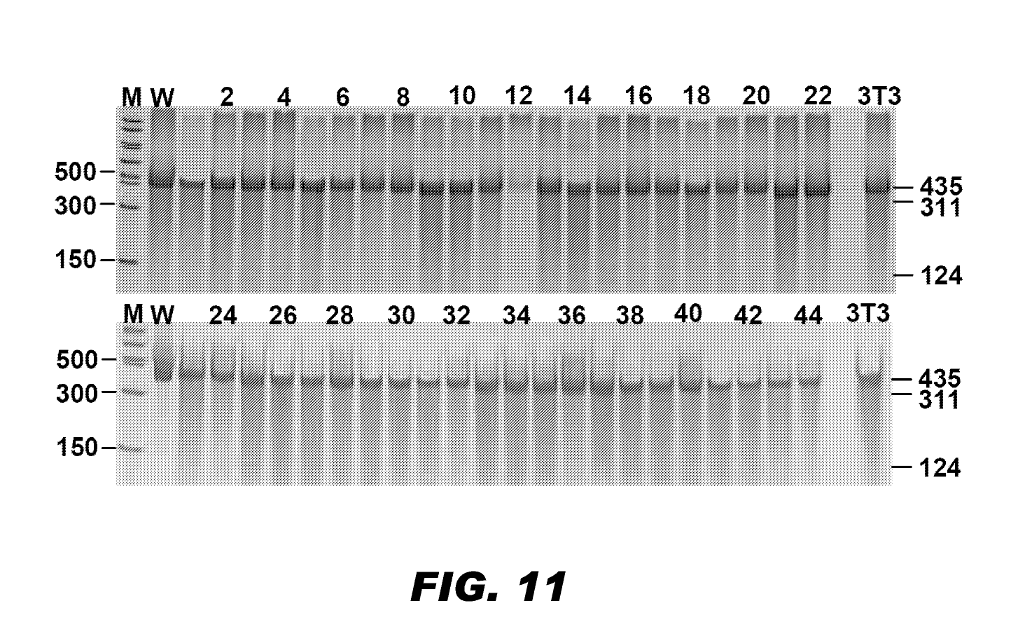

FIG. 11 presents the results of band isolation following isolation and purification of the species at the wild-type size in the Mdr1a−/− samples, and then use as a template in a nested PCR.

### DETAILED DESCRIPTION OF THE INVENTION

The present disclosure provides a genetically modified animal or animal cell comprising at least one edited chromosomal sequence encoding a protein involved in ADME and toxicology. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein involved in ADME and toxicology generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein involved in ADME and toxicology using targeted zinc finger nuclease technology is rapid, precise, and highly efficient.

### (I) Genetically Modified Animals

One aspect of the present disclosure provides a genetically modified animal in which at least one chromosomal sequence involved in ADME and toxicology has been edited. For example, the edited chromosomal sequence may be inactivated such that the sequence is not transcribed and/or a functional protein encoded by a chromosomal sequence involved in ADME and toxicology is not produced. Alternatively, the edited chromosomal sequence may be modified such that it codes for an altered protein involved in ADME and toxicology. For example, the chromosomal sequence may be modified such that at least one nucleotide is changed and the expressed protein involved in ADME and toxicology comprises at least changed amino acid residue. The modified protein involved in ADME and toxicology may have altered substrate specificity, altered enzyme activity, altered kinetic rates, and so forth. Furthermore, the edited chromosomal sequence encoding a protein involved in ADME and toxicology may comprise an integrated sequence and/or a sequence encoding an orthologous protein involved in ADME and toxicology that may be integrated into the genome of the animal. The genetically modified animal disclosed herein may be heterozygous for the edited chromosomal sequence involved in ADME and toxicology. Alternatively, the genetically modified animal may be homozygous for the edited chromosomal sequence involved in ADME and toxicology.

In one embodiment, the genetically modified animal may comprise at least one inactivated chromosomal sequence involved in ADME and toxicology. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional protein involved in ADME and toxicology is not produced. The inactivated chromosomal sequence comprises no exogenously introduced sequence. Such an animal may be termed a “knockout.” Also included herein are genetically modified animals in which two, three, or more chromosomal sequences involved in ADME and toxicology are inactivated.

In another embodiment, the genetically modified animal may comprise at least one chromosomally integrated sequence encoding an orthologous protein involved in ADME and toxicology or an endogenous protein involved in ADME and toxicology. For example, a sequence encoding an orthologous protein involved in ADME and toxicology may be integrated into a chromosomal sequence encoding a protein involved in ADME and toxicology such that the chromosomal sequence is inactivated, but wherein the exogenous sequence encoding the orthologous protein involved in ADME and toxicology may be expressed. In such a case, the sequence encoding the orthologous protein involved in ADME and toxicology may be operably linked to a promoter control sequence. Alternatively, a sequence encoding a protein involved in ADME and toxicology may be integrated into a chromosomal sequence without affecting expression of a chromosomal sequence. For example, a sequence encoding an orthologous protein involved in ADME and toxicology may be integrated into a “safe harbor” locus, such as the Rosa26 locus, HPRT locus, or AAV locus. In one iteration of the disclosure an animal, comprising a chromosomally integrated sequence encoding a protein involved in ADME and toxicology may be called a “knock-in,” and it should be understood that in such an iteration of the animal, no selectable marker is present. The present disclosure also encompasses genetically modified animals in which two, three, or more sequences encoding proteins involved in ADME and toxicology are integrated into the genome.

In an additional embodiment, the genetically modified animal may be a “humanized” animal comprising at least one chromosomally integrated sequence encoding a functional human protein involved in ADME and toxicology. The functional human ADME and toxicology-related protein may have no corresponding ortholog in the genetically modified animal. Alternatively, the wild-type animal from which the genetically modified animal is derived may comprise an ortholog corresponding to the functional human protein that is involved in ADME and toxicology. In this case, the orthologous sequence in the “humanized” animal is inactivated such that no functional protein is made and the “humanized” animal comprises at least one chromosomally integrated sequence encoding the human protein. Those of skill in the art appreciate that “humanized” animals may be generated by crossing a knock-out animal with a knock-in animal comprising the chromosomally integrated sequence.

In yet another embodiment, the genetically modified animal may comprise at least one edited chromosomal sequence encoding a protein involved in ADME and toxicology such that the expression pattern of the protein is altered. For example, regulatory regions controlling the expression of the protein, such as a promoter or transcription binding site, may be altered such that the protein involved in ADME and toxicology is over-produced, or the tissue-specific or temporal expression of the protein is altered, or a combination thereof. Alternatively, the expression pattern of the protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a protein, for example a protein involved in ADME and toxicology. The genetically modified animal comprising the lox-flanked chromosomal sequence encoding the protein may then be crossed with another genetically modified animal expressing Cre recombinase. Progeny animals comprising the lox-flanked chromosomal sequence and the Cre recombinase are then produced, and the lox-flanked chromosomal sequence encoding the protein involved in ADME and toxicology is recombined, leading to deletion or inversion of the chromosomal sequence encoding the protein. Expression of Cre recombinase may be temporally and conditionally regulated to effect temporally and conditionally regulated recombination of the chromosomal sequence encoding the protein of interest.

### (a) Chromosomal Sequences and Proteins Involved in ADME and Toxicology

Any chromosomal sequence or protein involved in ADME and toxicology may be utilized for purposes of the present invention. The ADME and toxicology-related proteins are typically selected based on an experimental association of the ADME and toxicology-related protein to an ADME and toxicology-related disorder. For example, the production rate or circulating concentration of an ADME and toxicology-related protein may be elevated or depressed in a population having an ADME and toxicology disorder relative to a population lacking the ADME and toxicology disorder. Differences in protein levels may be assessed using proteomic techniques including but not limited to Western blot, immunohistochemical staining, enzyme linked immunosorbent assay (ELISA), and mass spectrometry. Alternatively, the ADME and toxicology-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

Exemplary non-limiting examples of the chromosomal sequence or protein involved in ADME and toxicology may be chosen from Oct 1, Oct 2, Hfe2, Ppar(alpha), and combinations thereof, or from MDR1a (ABC Transporter ABCB1a), MDR1b (ABCB1b), BCRP (ABCC1), MRP1 (ABCG2) and MRP2 (ABCC2, cMOAT) and combinations thereof

(i) Oct 1 and Oct 2

Oct(organic cation transporter)-1 encodes an organic cation transporter predicted to contain twelve transmembrane domains. Oct-2 also encodes a cation transporter. Organic cations include endogenous compounds such as monoamine neurotransmitters, choline, and coenzymes, but also numerous drugs and xenobiotics.

(ii) Hfe2

Hfe2 (hemochromatosis type 2) provides instructions for making a protein called hemojuvelin. Hemojuvelin is a protein made in the liver, heart, and muscles that is used for movement as well as playing a role in maintaining iron balance in the body. The protein also regulates the levels of another protein called hepcidin. Hepcidin plays a key role in maintaining proper iron levels in the body. Hemochromatosis is caused by mutations in the Hfe2 gene and researchers have identified over 20 Hfe2 mutations that cause type 2 hemochromatosis. Most mutations in Hfe2 change one of the amino acids used to make hemojuvelin. Most frequently, the amino acid glycine is replaced by the amino acid valine at position 320 and other mutations can create a premature stop signal in the instructions for making hemojuvelin. The result of most of these mutations is a hemojuvelin protein that is abnormally small or one that does not function properly. In the absence of a working hemojuvelin protein, the levels of the protein hepcidin are reduced and iron balance is disturbed. This causes too much iron to be absorbed during digestion, leading to an iron overload that can damage tissues and organs in the body.

(iii) Ppar(alpha)

Ppar(alpha) (peroxisome proliferator-activated receptor alpha) is a nuclear receptor protein encoded by the PPARA gene. Peroxisome proliferators include hypolipidemic drugs, herbicides, leukotriene antagonists, and plasticizers. Peroxisome proliferators induce an increase in the size and number of peroxisomes, which are subcellular organelles found in plants and animals that contain enzymes for respiration and for cholesterol and lipid metabolism. PPARs are specific receptors believed to mediate peroxisome proliferators.

By way of example, the chromosomal sequence may comprise, but is not limited to, IL2 (interleukin 2), IL10 (interleukin 10), IL6 (interleukin 6 (interferon, beta 2)), IFNG (interferon, gamma), CYP3A4 (cytochrome P450, family 3, subfamily A, polypeptide 4), CYP2D6 (cytochrome P450, family 2, subfamily D, polypeptide 6), CYP1A2 (cytochrome P450, family 1, subfamily A, polypeptide 2), CYP1A1 (cytochrome P450, family 1, subfamily A, polypeptide 1), CASP3 (caspase 3, apoptosis-related cysteine peptidase), JUN (jun oncogene), GSTT1 (glutathione S-transferase theta 1), NR1I3 (nuclear receptor subfamily 1, group I, member 3), NOS1 (nitric oxide synthase 1 (neuronal)), ARNT (aryl hydrocarbon receptor nuclear translocator), CYP2B6 (cytochrome P450, family 2, subfamily B, polypeptide 6), NR1I2 (nuclear receptor subfamily 1, group I, member 2), GSTP1 (glutathione S-transferase pi 1), BCHE (butyrylcholinesterase), UGT1A1 (UDP glucuronosyltransferase 1 family, polypeptide A1), HSPA4 (heat shock 70 kDa protein 4), GSTM1 (glutathione S-transferase mu 1), NAT2 (N-acetyltransferase 2 (arylamine N-acetyltransferase)), and ABC transporters such as MDR1a (ABC Transporter ABCB1a), MDR1b (ABCB1b), BCRP (ABCC1), MRP1 (ABCG2) and MRP2 (ABCC2, cMOAT).

In a preferred embodiment, the edited chromosomal sequence(s) of the present invention is selected from Oct 1, Oct 2, Hfe2, Ppar(alpha), and combinations thereof. The edited chromosomal sequence(s) of the present invention may also comprise Oct 1 and at least one of Oct 2, Hfe2, and Ppar(alpha). The edited chromosomal sequence(s) of the present invention may also comprise Oct 2 and at least one of Oct 1, Hfe2, and Ppar(alpha). The edited chromosomal sequence(s) of the present invention may also comprise Hfe2 and at least one of Oct 1, Oct 2, and Ppar(alpha). The edited chromosomal sequence(s) of the present invention may also comprise Ppar(alpha) and at least one of Oct 1, Oct 2, and Hfe2. In an additionally preferred embodiment, the edited chromosomal sequence of the present invention comprises Oct 1, Oct 2, Hfe2, and Ppar(alpha). Table A details non-limiting examples of chromosomal sequences that may be edited in accordance with the present disclosure. For example, those rows having no entry in the “Protein Sequence” column indicate a genetically modified animal in which the sequence specified in that row under “Activated Sequence” is inactivated (i.e., a knock-out). Subsequent rows indicate single or multiple knock-outs with knock-ins of one or more integrated orthologous sequences, as indicated in the “Protein Sequence” column.

TABLE A

| Inactivated Sequence | Protein Sequence |
|---|---|
| oct 1 | none |
| oct 2 | none |
| hfe2 | none |
| Ppar(alpha) | noneoct 1 |
| OCT1 | |
| oct2 | OCT2 |
| hfe2 | HFE2 |
| Ppar(alpha) | PPAR(alpha) |
| oct 1, oct 2 | OCT 1, OCT2 |
| oct 1, hfe2 | OCT 1, HFE2 |
| oct 1, ppar(alpha) | OCT 1, PPAR(alpha) |
| oct 2, hfe2 | OCT 2, HFE2 |
| oct 2, ppar(alpha) | OCT 2, PPAR(alpha) |
| hfe2, ppar(alpha) | HFE2, PPAR(alpha) |
| oct 1, oct 2, hfe2 | OCT 1, OCT 2, HFE2 |
| oct 1, oct 2, ppar(alpha) | OCT 1, OCT 2, PPAR(alpha) |
| oct 2, hfe2, ppar(alpha) | OCT 2, HFE2, PPAR(alpha) |
| oct 1, hfe2, ppar(alpha) | OCT 1, HFE2, PPAR(alpha) |
| oct 1, oct 2, hfe2, ppar(alpha) | OCT 1, OCT 2, HFE2, PPAR(alpha) |

### (b) Animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. In another iteration of the invention, the animal does not comprise a genetically modified mouse. In each of the foregoing iterations of suitable animals for the invention, the animal does not include exogenously introduced, randomly integrated transposon sequences.

### (c) Protein Involved in ADME and Toxicology

The protein involved in ADME and toxicology may be from any of the animals listed above. Furthermore, the protein involved in ADME and toxicology may be a human protein. Additionally, the protein involved in ADME and toxicology may be a bacterial, fungal, or plant protein. The type of animal and the source of the protein can and will vary. As an example, the genetically modified animal may be a rat, cat, dog, or pig, and the orthologous protein involved in ADME and toxicology may be human. Alternatively, the genetically modified animal may be a rat, cat, or pig, and the orthologous protein involved in ADME and toxicology may be canine. One of skill in the art will readily appreciate that numerous combinations are possible.

### (II) Genetically Modified Cells

A further aspect of the present disclosure provides genetically modified cells or cell lines comprising at least one edited chromosomal sequence involved in ADME and toxicology. The genetically modified cell or cell line may be derived from any of the genetically modified animals disclosed herein. Alternatively, the chromosomal sequence involved in ADME and toxicology may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Manassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

In a preferred embodiment the chromosomal sequence may be targeted for editing in any of the following commonly used rat strains: Dahl Salt-Sensitive, Fischer 344, Lewis, Long Evans Hooded, Sprague-Dawley, or Wistar.

Additionally, a gene encoding a protein involved in ADME and toxicology may be modified to include a tag or reporter gene or genes as are well-known. Reporter genes include those encoding selectable markers such as chloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### (III) Zinc Finger-Mediated Genome Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; and Choo et al. (2000) Curr. Opin. Struct. Biol. 10:411-416. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (see, for example, Biochemistry 2002, 41, 7074-7081).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence that facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a FokI cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from I to K in one cleavage monomer to produce an engineered cleavage monomer designated “E490K:I538K” and by mutating positions 486 from Q to E and 499 from I to L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences involved in ADME and toxicology may further comprise introducing at least one donor polynucleotide comprising a sequence encoding a protein involved in ADME and toxicology into the embryo or cell. A donor polynucleotide comprises at least three components: the sequence coding the protein involved in ADME and toxicology, an upstream sequence, and a downstream sequence. The sequence encoding the protein is flanked by the upstream and downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding a protein involved in ADME and toxicology may be a BAC.

The sequence of the donor polynucleotide that encodes the protein involved in ADME and toxicology may include coding (i.e., exon) sequence, as well as intron sequences and upstream regulatory sequences (such as, e.g., a promoter). Depending upon the identity and the source of the protein involved in ADME and toxicology, the size of the sequence encoding the protein involved in ADME and toxicology can and will vary. For example, the sequence encoding the protein involved in ADME and toxicology may range in size from about 1 kb to about 5,000 kb.

The donor polynucleotide also comprises upstream and downstream sequences flanking the chromosomal sequence involved in ADME and toxicology. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a chromosomal sequence involved in ADME and toxicology, a double stranded break introduced into the chromosomal sequence by the zinc finger nuclease is repaired, via homologous recombination with the donor polynucleotide, such that the chromosomal sequence involved in ADME and toxicology is integrated into the chromosome. The presence of a double-stranded break facilitates integration of the sequence into the chromosome. A donor polynucleotide may be physically integrated or, alternatively, the donor polynucleotide may be used as a template for repair of the break, resulting in the introduction of the chromosomal sequence involved in ADME and toxicology as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences involved in ADME and toxicology may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 bp to about 10,000 bp in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 bp in length. In other embodiments, the sequence in the exchange polynucleotide may be about 5500, 6000, 6500, 6000, 6500, 7000, 7500, 8000, 8500, 9000, 9500, or 10,000 bp in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking, the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence involved in ADME and toxicology in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion, or nonsense mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated Oct 1 chromosomal sequence may be crossed with animal B comprising a chromosomally integrated sequence encoding a human Oct 1 protein to give rise to a “humanized” Oct 1 offspring comprising both the inactivated Oct1 chromosomal sequence and the chromosomally integrated human Oct 1 sequence. Similarly, an animal comprising an inactivated Hfe2 chromosomal sequence may be crossed with an animal comprising a chromosomally integrated sequence encoding the human Hfe2 protein to generate “humanized” Hfe2 offspring. Moreover, a humanized Oct 1 animal may be crossed with a humanized Hfe2 animal to create a humanized Oct 1/Hfe2 animal. Those of skill in the art will appreciate that many combinations are possible.

In other embodiments, an animal comprising an edited chromosomal sequence disclosed herein may be crossbred to combine the edited chromosomal sequence with other genetic backgrounds. By way of non-limiting example, other genetic backgrounds may include wild-type genetic backgrounds, genetic backgrounds with deletion mutations, genetic backgrounds with another targeted integration, and genetic backgrounds with non-targeted integrations. Suitable integrations may include, without limit, nucleic acids encoding drug transporter proteins, Mdr protein, and the like.

### (IV) Applications

A further aspect of the present disclosure encompasses a method for assessing the effect(s) of an agent. Suitable agents include without limit pharmaceutically active ingredients, drugs, food additives, pesticides, herbicides, toxins, industrial chemicals, household chemicals, and other environmental chemicals. For example, the effect(s) of an agent may be measured in a “humanized” genetically modified animal, such that the information gained therefrom may be used to predict the effect of the agent in a human. In general, the method comprises contacting a genetically modified animal comprising at least one inactivated chromosomal sequence involved in ADME and toxicology and at least one chromosomally integrated sequence encoding an orthologous protein involved in ADME and toxicology with the agent, and comparing results of a selected parameter to results obtained from contacting a wild-type animal with the same agent. Selected parameters include but are not limited to (a) rate of elimination of the agent or its metabolite(s); (b) circulatory levels of the agent or its metabolite(s); (c) bioavailability of the agent or its metabolite(s); (d) rate of metabolism of the agent or its metabolite(s); (e) rate of clearance of the agent or its metabolite(s); (f) toxicity of the agent or its metabolite(s); (g) efficacy of the agent or its metabolite(s); (h) disposition of the agent or its metabolite(s); and (i) extrahepatic contribution to metabolic rate and clearance of the agent or its metabolite(s).

Also provided are methods to assess the effect(s) of an agent in an isolated cell comprising at least one edited chromosomal sequence p involved in ADME and toxicology, as well as methods of using lysates of such cells (or cells derived from a genetically modified animal disclosed herein) to assess the effect(s) of an agent. For example, the role of a particular protein involved in ADME and toxicology in the metabolism of a particular agent may be determined using such methods. Similarly, substrate specificity and pharmacokinetic parameter may be readily determined using such methods. Those of skill in the art are familiar with suitable tests and/or procedures.

Yet another aspect encompasses a method for assessing the therapeutic efficacy of a potential gene therapy strategy. That is, a chromosomal sequence encoding a protein of interest, such as a protein involved in ADME and toxicology, may be modified such that an undesired ADME characteristic or toxic effect is reduced or eliminated. In particular, the method comprises editing a chromosomal sequence encoding a protein of interest, such as a protein involved in ADME and toxicology, such that an altered protein product is produced. The genetically modified animal may be further exposed to test conditions and behavioral, cellular, and/or molecular responses measured and compared to those of a wild-type animal exposed to the same test conditions. Consequently, the therapeutic potential of the gene therapy regime may be assessed.

Still yet another aspect encompasses a method of generating a cell line or cell lysate using a genetically modified animal comprising an edited chromosomal sequence encoding a protein of interest, such as a protein involved in ADME and toxicology. An additional other aspect encompasses a method of producing purified biological components using a genetically modified cell or animal comprising an edited chromosomal sequence encoding a protein of interest, such as a protein involved in ADME and toxicology. Non-limiting examples of biological components include antibodies, cytokines, signal proteins, enzymes, receptor agonists and receptor antagonists.

Among the proteins of interest that are involved in drug ADME and toxicology are the ABC transporters, also known as efflux transport proteins. Thus, for example, the genetically modified animals as described herein containing an edited chromosomal sequences encoding an ABC transporter can be useful for screening biologically active agents including drugs and for investigating their distribution, efficacy, metabolism and/or toxicity. These screening methods are of particular use for assessing with improved predictability the behavior of a drug in a genetically modified animal as described herein, e.g. in a genetically modified rat, as a model for humans. Accordingly, the present disclosure also features a method of assessing the ADME profile of a drug in a genetically modified animal, as part of a drug screening or evaluation process. A candidate therapeutic agent, i.e, a candidate drug can be administered to a genetically modified animal that harbors a targeted gene knockout out and/or an expressed transgene, which was achieved through use of ZFNs. The knock-out or knock-in is associated with at least one aspect of the drug ADME profile or toxicology, and/or metabolism, and is expressed naturally in mouse, or rat, or human.

For example, a method of screening for the target of a test compound can make use of a genetically modified animal in which any one or more of an ABC transporter such as Mdr1a, Mdr1b, PXR, BCRP, MRP1, or MRP2 are knocked out, thus inhibiting or eliminating transmembrane transport mediated by the knocked out protein(s). Such an animal can be exposed to a test compound suspected of inhibiting transporter activity of the knocked-out protein(s). Inhibition of transport by the compound in the genetically modified animal can be determined using any of a number of routine laboratory tests and techniques, and the inhibition of transport compared to that observed in a wild-type animal treated with the same test compound. A difference in the effect of the test compound in the two animals can be indicative of the target of the test compound. Further, inhibition of one or more ABC transporter proteins such as Mdr1a, Mdr1b, PXR, BCRP, MRP1, or MRP2, may improve certain ADME characteristics of a candidate therapeutic agent. For example, the absorption or efficacy of a candidate therapeutic compound may be improved by knocking out expression of one or more ABC transporter proteins such as Mdr1a, Mdr1b, PXR, BCRP, MRP1, or MRP2, in a particular tissue. It will thus be understood that genetically modified animals and cells as described herein, for example genetically modified animals and cells including a genetic modification of one or more ABC transporter proteins, can be used advantageously in many methods that evaluate the ADME and toxicology characteristics of a candidate therapeutic compound, to identify targets of a test compound, or to identify ways in which the ADME characteristics and toxicology of a candidate compound may be improved.

The overwhelming need to accurately predict how drugs and environmental chemicals will affect large populations can be readily appreciated. The genetically modified animals, embryos, cells and cell lines described herein can be used to analyze how various compounds will interact with biological systems. Genetically modified cells and cell lines, can be used, for example, to control many of the known complexities in biological systems to improve the predictive ability of cell-based assay systems, such as those that may be used to evaluate new molecular entities and possible drug-drug interactions. More specifically, it is recognized that biological systems include multiple components that respond to exposure to new, potentially harmful compounds.

The “ADMET system” has been described as including five components. The first component are those biological systems, which when disrupted, signal the drug metabolism system to turn on. These can be any of the many stress response and DNA repair pathways. Once “on”, the next components of the system, the “xenosensors”, begin to surveil for exogenous molecules that need removal. Detection by the xenosensors then activates a cascade of gene inductions that up-regulate the enzymes responsible for metabolizing exogenous molecules into forms for easier removal. The third component is made up of Phase I enzymes that are composed of at least three classes of oxidases, of which the best known class is the cytochrome P450s. These tend to add reactive hydroxyl moieties to potential toxins, inactivating them, and making them more polar (soluble). The fourth component of the system is composed of at least seven classes of enzymes that further alter the products from Phase I modification. Typically, these are conjugating enzymes that add hydrophilic moieties to make the now oxidized xenobiotics even more water soluble, and therefore, more readily collected and excreted through urine or bile. The last component is the transporter system involving the transporter proteins, such as the ABC transporters among other classes, that function as molecular pumps to facilitate the movement of the xenobiotics from one tissue to another. They are responsible for moving drugs into a cell, out of a cell, or through a cell. Each member of the different components of the ADMET system has its own set of substrate structural specificities, which must be taken into account by any assay. Making predictability an even larger challenge is that, for critical members of each of the five component classes, a constellation of genetic polymorphisms exists in the population and these can dramatically affect activity towards specific xenobiotic chemical structures. The growing field of pharmacogenomics addresses the challenges created by such genetic variation. In addition, gender differences in how the different components of the xenobiotic system respond are also known to play a role in variations in drug metabolism. Thus, genetically modified animals, cells and particularly cell lines as described herein will be useful as the basis for cell-based assays with improved predictive ability with respect to a drug's clinical outcome or a chemical's toxicological problems. Panels of cell lines are expressly contemplated for such a purpose. For example, cell-based assays can be created that are representative of the target tissue where metabolism or toxicity of a test, e.g., a drug compound is likely to occur. Presently, standard assays are usually run in transformed cell lines that are derived from the target tissue and have some concordant functional properties. To create even better cell-based assays that are even more representative of the natural state, genetically modified and differentiated pluripotent cells could be used to replace the immortalized cell components. In other words, genetically modified cell lines can be used in more highly predictive cell-based assays suitable for high-throughput, high-content compound screening.

Accordingly, the present disclosure contemplates ZFN-mediated genetic modifications of genes relevant to each part of the xenobiotic metabolism machinery. Such modifications include knockouts, knock-ins of reporter tags, the introduction of specific mutations known to affect activity, or combinations of these. For example, the genetically modified cells and cell lines can be used to create tissue-specific, gender-specific, population-reflective, transporter panels; cell-based xenosensor assay panels that are tissue specific and functionally reflective of the population; and induction assays that measure the genetic activation of different drug metabolism components and overt toxicological responses such as genotoxicity, cardiotoxicity, and apoptosis.

According to the present disclosure, tissue-specific lines can be established that have been modified to isolate specific transporter activities and predict the reaction of populations to individual chemical entities. For example, ZFNs can be used to create transporter gene knockouts in enterocyte cell lines, such as to introduce important, common polymorphisms into enterocyte cell lines, and in cell lines representative of liver, blood-brain-barrier (brain micro-vasculature endothelial cells), kidney and any relevant tissue-specific cell lines. Panels of cell lines can include enterocytes (Caco2 or BBe1) with knock-outs of individual transporter proteins (e.g. MDR-1, MRP1, 2, 3, 4, 6, BCRP), knockout combinations to isolate effects of individual transporters (e.g. BCRP and MRP2, MDR-1 and MRP2, MRP-3 and MRP1), and a transporter null line (i.e. all 7 transporters knocked out]). Panels of enterocytes may include knock-outs of OATP-2B1, PEPT-1, and OCT-N2. Panels of enterocytes may created which include prevalent polymorphisms in the major transporter genes that affect drug transport and are of concern to pharmaceutical companies.

Still further, the three xenosensors in humans (PXR, AhR and CAR) have overlapping specificities with response to xenobiotics. Knowing which are activated, and to what extent, by any particular chemical compound is also an important consideration for understanding drug response, and drug-drug interactions. Creating panels of cells that report induction by the xenosensors can delineate the specificities. Further modifying the cells to address functionally important polymorphisms in the xenosensors would permit desperately needed population predictions. ZFNs can be used to create knockout cell lines analogous to transporter knock-out cell lines as described above, and to create reporter cell lines that express different fluorescent proteins upon induction of different xenosensors. For example, cell lines in which green FP is expressed if PXR is induced, red FP if CAR activity is induced, blue FP if AhR is induced. These can be constructed in the relevant tissue-type cell lines, i.e. intestine, liver, kidney, brain, and heart. Panels of cells can be created that represent the tissues most involved with drug toxicity and metabolism, and in which each xenosensor (CAR, PXR, AhR) is knocked out. Cell lines can also be produced that produce fluorescent proteins upon the activation of each of the three xenosensors.

Also contemplated are induction assays of ADME biotransformation and toxicological response genes. While the activities of each of the many Phase I and Phase II enzymes can be done today in simple biochemical assays, available assays cannot measure, in high-throughput fashion, the induction of any particular enzyme by an exogenously added xenobiotic. ZFNs can be used to create genetically modified cell lines as described herein that can provide the basis for assays that can measure the up/down regulation of key Phase I and Phase II enzymes, along with genes involved in a toxicological response. For example, ZFNs can be used to build lines that have a reporter protein (e.g. fluorescent protein or luciferase) gene inserted proximal to the promoter of the gene being measured. These gene targets can be any of the critical Phase I, Phase II, transporter, genotox, or apoptosis/necrosis pathway components. Tissue-specific panels of cells can also be created, which report on the activation of genes encoding either the Phase I or Phase II enzymes, the transporters, or toxicity response pathways (e.g., genotoxicity or apoptosis).

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “chromosomal sequence involved in ADME and toxicology” refers to a chromosomal sequence that has been identified to contribute to or be involved in the process of absorption, distribution, metabolism, excretion (ADME), and/or toxicology. ADME and toxicology affect the disposition of a pharmaceutical compound within an organism. Exemplary chromosomal sequences involved in ADME and toxicology include, but are not limited to, Oct 1, Oct 2, Hfe2, and Ppar(alpha). Any chromosomal sequence known to be involved in ADME and toxicology will work for purposes of the present invention.

The term “a protein encoded by a chromosomal sequence involved in ADME and toxicology” or “a protein involved in ADME and toxicology” refers to a protein that has been encoded by a chromosomal sequence identified to contribute to or be involved in the process of absorption, distribution, metabolism, excretion (ADME), and/or toxicology. Exemplary proteins involved in ADME and toxicology include, but are not limited to, organic cation transporter 1, a protein encoded by Oct 1; organic transporter cation 2, a protein encoded by Oct 2; hemojuvelin, a protein encoded by Hfe2; and peroxisome proliferator-activated receptor alpha, a nuclear receptor protein encoded by Ppar(alpha). Any type of protein involved in ADME and toxicology will work for purposes of the present invention including, but not limited to, structural proteins, enzyme and catalytic proteins, transport proteins, hormonal proteins, contractile proteins, storage proteins, genetic proteins, defense proteins, and receptor proteins.

A “gene,” as used herein, refers to a DNA region (including exons and introns) encoding a gene product, as well as all DNA regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of Oct 1 in a Model Organism

ZFN-mediated genome editing may be used to study the effects of a “knockout” mutation in an AD-related chromosomal sequence, such as a chromosomal sequence encoding the Oct 1 protein, in a genetically modified model animal and cells derived from the animal. Such a model animal may be a rat. In general, ZFNs that bind to the rat chromosomal sequence encoding the Oct 1 protein associated with AD may be used to introduce a deletion or insertion such that the coding region of the Oct 1 gene is disrupted such that a functional Oct 1 protein may not be produced.

Suitable fertilized embryos which may be at the single-cell stage may be microinjected with capped, polyadenylated mRNA encoding the ZFN. The frequency of ZFN-induced double strand chromosomal breaks may be determined using the Cel-1 nuclease assay, as detailed above. The sequence of the edited chromosomal sequence may be analyzed as described above. The development of AD symptoms and disorders caused by the Oct 1 “knockout” may be assessed in the genetically modified rat or progeny thereof. Furthermore, molecular analyses of AD-related pathways may be performed in cells derived from the genetically modified animal comprising an ErbB4 “knockout”.

### Example 2

### Generation of a Humanized Rat Expressing a Mutant Form of Human Genes Involved in ADME and Toxicology

Mutations in any of the chromosomal sequences involved in ADME and toxicology can be used in the generation of a humanized rat expressing a mutant form of the gene. The genes can be Oct 1, Oct 2, Hfe2, Ppar(alpha), and combinations thereof. ZFN-mediated genome editing may be used to generate a humanized rat wherein the rat gene is replaced with a mutant form of the human gene comprising the mutation. Such a humanized rat may be used to study the development of the diseases associated with the mutant human protein encoded by the gene of interest. In addition, the humanized rat may be used to assess the efficacy of potential therapeutic agents targeted at the pathway leading to AD comprising the gene of interest.

The genetically modified rat may be generated using the methods described in the Example above. However, to generate the humanized rat, the ZFN mRNA may be co-injected with the human chromosomal sequence encoding the mutant protein into the rat embryo. The rat chromosomal sequence may then be replaced by the mutant human sequence by homologous recombination, and a humanized rat expressing a mutant form of the protein may be produced.

### Example 3

### Identification of ZFNs that Edit the Mdr1a Locus

The Mdr1a gene was chosen for zinc finger nuclease (ZFN) mediated genome editing. ZFNs were designed, assembled, and validated using strategies and procedures previously described (see Geurts et al., Science (2009) 325:433). ZFN design made use of an archive of pre-validated 1-finger and 2-finger modules. The rat Mdr1a gene region (NM_{—}133401) was scanned for putative zinc finger binding sites to which existing modules could be fused to generate a pair of 4-, 5-, or 6-finger proteins that would bind a 12-18 bp sequence on one strand and a 12-18 bp sequence on the other strand, with about 5-6 bp between the two binding sites.

Capped, polyadenylated mRNA encoding each pair of ZFNs was produced using known molecular biology techniques. The mRNA was transfected into rat cells. Control cells were injected with mRNA encoding GFP. Active ZFN pairs were identified by detecting ZFN-induced double strand chromosomal breaks using the Cel-1 nuclease assay. This assay detects alleles of the target locus that deviate from wild type as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and re-annealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay revealed that the ZFN pair targeted to bind 5′-acAGGGCTGATGGCcaaaatcacaagag-3′ (SEQ ID NO: 8; contact sites in uppercase) and 5′-ttGGACTGTCAGCTGGTatttgggcaaa-′3′ (SEQ ID NO: 9) cleaved within the Mdr1a locus.

### Example 4

### Editing the Mdr1a Locus

Capped, polyadenylated mRNA encoding the active pair of ZFNs was microinjected into fertilized rat embryos using standard procedures (e.g., see Geurts et al. (2009) supra). The injected embryos were either incubated in vitro, or transferred to pseudopregnant female rats to be carried to parturition. The resulting embryos/fetus, or the toe/tail clip of live born animals were harvested for DNA extraction and analysis. DNA was isolated using standard procedures. The targeted region of the Mdr1a locus was PCR amplified using appropriate primers. The amplified DNA was subcloned into a suitable vector and sequenced using standard methods. FIG. 1 presents DNA sequences of edited Mdr1a loci in two animals. One animal had a 20 bp deletion in the target sequence in exon 7, and a second animal had a 15 bp deletion and a 3 bp insertion in the target sequence of exon 7. The edited loci harbored frameshift mutations and multiple translational stop codons.

Western analyses were performed to confirm that the Mdr1a locus was inactivated such that no Mdr1a protein was produced. A cell lysate was prepared from the proximal colon of Mdr1a knockout rat. Control cell lysate was prepared from a human neuroblastoma cell line. As shown on FIG. 2, no Mdr1a protein was detected in the Mdr1a (−/−) animal, indicating that the Mdr1a locus was inactivated.

### Example 5

### Identification of ZFNs that Edit the Mdr1b Locus

ZFNs that target and cleave the Mdr1b gene were identified essentially as described above. The rat Mdr1b gene (NM_{—}012623) was scanned for putative zinc finger binding sites. ZFNs were assembled and tested essentially as described in Example 1. This assay revealed that the ZFN pair targeted to bind 5′-agGAGGGGAAGCAGGGTtccgtggatga-3′ (SEQ ID NO: 10; contact sites in uppercase) and 5′-atGCTGGTGTTCGGatacatgacagata-3′ (SEQ ID NO: 11) cleaved within the Mdr1b locus.

### Example 6

### Identification of ZFNs that Edit the Mrp1 Locus

ZFNs that target and cleave the Mrp1 gene were identified essentially as described above in Example 1. The rat Mrp1 gene (NM_{—}022281) was scanned for putative zinc finger binding sites. ZFNs were assembled and tested essentially as described in Example 1. This assay revealed that the ZFN pair targeted to bind 5′-gaAGGGCCCAGGTTCTAagaaaaagcca-3′ (SEQ ID NO: 12; contact sites in uppercase) and 5′-tgCTGGCTGGGGTGGCTgttatgatcct-′3′ (SEQ ID NO: 13) cleaved within the Mrp1 locus.

### Example 7

### Editing the Mrp1 Locus

Rat embryos were microinjected with mRNA encoding the active pair of Mrp1 ZFNs essentially as described in Example 2. The injected embryos were incubated and DNA was extracted from the resultant animals. The targeted region of the Mrp1 locus was PCR amplified using appropriate primers. The amplified DNA was subcloned into a suitable vector and sequenced using standard methods. FIG. 3 presents DNA sequences of edited Mrp1 loci in two animals. One animal had a 43 bp deletion in exon 11, and a second animal had a 14 bp deletion in exon 11. These deletions disrupt the reading frame of the Mrp1 coding region.

### Example 8

### Identification of ZFNs that Edit the Mrp2 Locus

ZFNs that target and cleave the Mrp2 gene were identified essentially as described above in Example 1. The rat Mrp2 gene (NM_{—}012833) was scanned for putative zinc finger binding sites. ZFNs were assembled and tested essentially as described in Example 1. This assay revealed that the ZFN pair targeted to bind 5′-ttGCTGGTGACtGACCTTgttttaaacc-3′ (SEQ ID NO: 14; contact sites in uppercase) and 5′-ttGAGGCGGCCATGACAAAGgacctgca-′3′ (SEQ ID NO: 15) cleaved within the Mrp2 locus.

### Example 9

### Editing the Mrp2 Locus

Rat embryos were microinjected with mRNA encoding the active pair of Mrp2 ZFNs essentially as described in Example 2. The injected embryos were incubated and DNA was extracted from the resultant animals. The targeted region of the Mrp2 locus was PCR amplified using appropriate primers. The amplified DNA was subcloned into a suitable vector and sequenced using standard methods. FIG. 4 presents DNA sequence of an edited Mrp2 locus in which 726 bp was deleted from exon 7, thereby disrupting the reading frame of the Mrp2 coding region.

### Example 10

### Identification of ZFNs that Edit the BCRP Locus

ZFNs that target and cleave the BCRP gene were identified essentially as described above in Example 1. The rat BCRP gene (NM_{—}181381) was scanned for putative zinc finger binding sites. ZFNs were assembled and tested essentially as described in Example 1. This assay revealed that the ZFN pair targeted to bind 5′-atGACGTCAAGGAAGAAgtctgcagggt-3′ (SEQ ID NO: 16; contact sites in uppercase) and 5′-acGGAGATTCTTCGGCTgtaatgttaaa-′3′ (SEQ ID NO: 17) cleaved within the BCRP locus.

### Example 11

### Editing the BCRP Locus

Rat embryos were microinjected with mRNA encoding the active pair of BCRP ZFNs essentially as described in Example 2. The injected embryos were incubated and DNA was extracted from the resultant animals. The targeted region of the BCRP gene was PCR amplified using appropriate primers. The amplified DNA was subcloned into a suitable vector and sequenced using standard methods. FIG. 5 presents the DNA sequences of edited BCRP loci in two founder animals. One animal had a 588 bp deletion in exon 7, and the second animal had a 696 bp deletion in exon 7. These deletions disrupt the reading frame of the BCRP coding region.

### Example 12

### Disruption of Mdr1a

In vitro preparation of ZFN mRNAs: the ZFN expression plasmids were obtained from Sigma's CompoZr product line. Each plasmid was linearized at the XbaI site, which is located at the 3′ end of the FokI ORF. 5′ capped and 3′ polyA tailed message RNA was prepared using either MessageMax T7 Capped transcription kit and poly (A) polymerase tailing kit (Epicentre Biotechnology, Madison, Wis.) or mMessage Machine T7 kit and poly (A) tailing kit (Ambion, Austin, Tex.). The poly A tailing reaction was precipitated twice with an equal volume of 5 M NH4OAc and then dissolved in injection buffer (1 mM Tris-HCl, pH 7.4, 0.25 mM EDTA). mRNA concentration was estimated using a NanoDrop 2000 Spectrometer (Thermo Scientific, Wilmington, Del.).

ZFN validation in cultured cells: In short, when ZFNs make a double-strand break at the target site that is repaired by the non-homologous end-joining pathway, deletions or insertions are introduced. The wild-type and mutated alleles are amplified in the same PCR reaction. When the mixture is denatured and allowed to re-anneal, the wild-type and mutated alleles form double strands with unpaired region around the cleavage site, which can be recognized and cleaved by a single strand specific endonuclease to generate two smaller molecules in addition to the parental PCR product. The presence of the cleaved PCR bands indicates ZFN activity in the transfected cells.

The NIH 3T3 cells were grown in DMEM with 10% FBS and antibiotics at 37° C. with 5% CO2. ZFN mRNAs were paired at 1:1 ratio and transfected into the NIH 3T3 cells to confirm ZFN activity using a Nucleofector (Lonza, Basel, Switzerland), following the manufacture's 96-well shuttle protocol for 3T3 cells. Twenty-four hours after transfection, culturing medium was removed, and cells were incubated with 15 ul of trypsin per well for 5 min at 37° C. Cell suspension was then transferred to 100 ul of QuickExtract (Epicentre) and incubated at 68° C. for 10 min and 98° C. for 3 min. The extracted DNA was then used as template in a PCR reaction to amplify around the target site with following primer pairs:

Mdr1a Cel-I F:
(SEQ ID NO: 18)
ctgtttcttgacaaaacaacactaggctc
Mdr1a Cel-I R:
(SEQ ID NO: 19)
gggtcatgggaaagagtttaaaatc

Each 50 ul PCR reaction contained 1 ul of template, 5 ul of buffer II, 5 ul of 10 uM each primer, 0.5 ul of AccuPrime High Fidelity (Invitrogen, Carlsbad, Calif.) and 38.5 ul of water. The following PCR program was used: 95° C., 5 min, 35 cycles of 95° C., 30 sec, 60° C., 30 sec, and 68° C., 45 sec, and then 68° C., 5 min, 4° C. Three microliter of the above PCR reaction was mixed with 7 ul of 1× buffer II and incubated under the following program: 95° C., 10 min, 95° C. to 85° C., at −2° C./s, 85° C. to 25° C. at −0.1° C./s, 4° C. forever One microliter each of nuclease S and enhancer (Transgenomic, Omaha, Nebr.) were added to digest the above reaction at 42° C. for 20 min. The mixture is resolved on a 10% polyacrylamide TBE gel (Bio-Rad, Hercules, Calif.).

Microinjection and mouse husbandry: FVB/NTac and C57BL/6NTac mice were housed in static cages and maintained on a 14 h/10 h light/dark cycle with ad libitum access to food and water. Three to four week-old females were injected with PMS (5 I.U./per mouse) 48 h before hCG (5 I.U./mouse) injection. One-cell fertilized eggs were harvested 10-12 h after hCG injection for microinjection. ZFN mRNA was injected at 2 ng/ul. Injected eggs were transferred to pseudopregnant females (Swiss Webster (SW) females from Taconic Labs mated with vasectomized SW males) at 0.5 dpc.

Founder identification using mutation detection assay: toe clips were incubated in 100-200 ul of QuickExtract (Epicentre Biotechnology) at 50° C. for 30 min, 65° C. for 10 min and 98° C. for 3 min. PCR and mutation detection assay were done under the same conditions as in ZFN validation in cultured cells using the same sets of primers.

TA cloning and sequencing: to identify the modifications in founders, the extracted DNA was amplified with Sigma's JumpStart Taq ReadyMix PCR kit. Each PCR reaction contained 25 ul of 2× ReadyMix, 5 ul of primers, 1 ul of template, and 19 ul of water. The same PCR program was used as in ZFN validation in cultured cells. Each PCR reaction was cloned using TOPO TA cloning kit (Invitrogen) following the manufacture's instructions. At least 8 colonies were picked from each transformation, PCR amplified with T3 and T7 primers, and sequenced with either T3 or T7 primer. Sequencing was done at Elim Biopharmaceuticals (Hayward, Calif.).

PCR for detecting large deletions: to detect larger deletions, another set of primers were used for each of the target:

Mdr1a 800F:
(SEQ ID NO: 20)
catgctgtgaagcagatacc
Mdr1a 800R:
(SEQ ID NO: 21)
ctgaaaactgaatgagacatttgc

Each 50 ul PCR contained: 1 ul of template, 5 ul of 10× buffer II, 5 ul of 10 uM of each 800 F/R primer, 0.5 ul of AccuPrime Taq Polymerase High Fidelity (Invitrogen), and 38.5 ul of water. The following program was used: 95° C., 5 min, 35 cycles of 95° C., 30 sec, 62° C., 30 sec, and 68° C., 45 sec, and then 68° C., 5 min, 4° C., forever. The samples were resolved on a 1% agarose gel. Distinct bands with lower molecular weight than the wt were sequenced.

RNA preparation from tissues and RT-PCR: Mdr1a−/− or Mdr1a+/+ littermates were sacrificed for tissue harvest at 5-9 weeks of age. Large intestine, kidney and liver tissues were dissected and immediately used or archived for later processing, tissue biopsies were placed in RNAlater solution (Ambion) and stored at −20° C. Total RNA was prepared using GenElute Mammalian Total RNA Miniprep kit (Sigma) following manufacture's instructions. To eliminate any DNA contamination the RNA was treated with DNAseI (New England Biolabs, Ipswich, Mass.) before being loaded onto the purification columns. RT-PCR reaction was carried out with 1 ul of total RNA, primers RT-F (5′-GCCGATAAAAGAGCCATGTTTG) (SEQ ID NO: 22) and RT-R (5′-GATAAGGAGAAAAGCTGCACC) (SEQ ID NO: 23), using SuperScript™ III One-Step RT-PCR System with Platinum® Taq High Fidelity kit (Invitrogen). Reverse transcription and subsequent PCR were carried out with 1 cycle of 55° C. for 30 min. and 94° C. for 2 min. for cDNA synthesis; and 40 cycles of 94° C. for 15 sec, 56° C. for 30 sec, and 68° C. for 1 min for amplification. The PCR product was loaded in a 1.2% agarose gel and visualized with ethidium bromide.

TABLE 1
Summary of deletions in Mdr1a
−10 −5 −2 +2 +5 +10
GCCATCAGCCCTGTTICTTGGACTGTCAGCTGGT

| | Deletion size | |
|---|---|---|
| ID | (bp)+ insertion | Position |
| 2 | 6 + A | −4, +2 |
| 3 | 4 + C | −1, +3 |
| 4 | 3 | −2, +1 |
| 5 | 646 | −640, +6 |
| 6 | 695 | −583, +112 |
| 7 | 19 | −14, +5 |
| 8 | 248 | −238, +10 |
| 11 | 417, 19 | (−528-−112),(-14, +5) |
| | 533 | −27, +506 |
| 13 | 392 | −20, +372 |
| 17 | 2 | −1, +1 |
| | 19 | −14, +5 |
| | 19 | −18, +1 |
| 18 | 2 | +1-+2 |
| 19 | 25 | −25- -1 |
| 20 | 19 | −15, +6 |
| 21 | 533 | −524, +9 |
| | 584 | −579, +5 |
| 23 | 396 | −389, +7 |
| 25 | 533 | −6, +527 |
| 26 | 13 | −5, +8 |
| | 534 | −516, +18 |
| 27 | 75 | −72, +3 |
| | 19 | −14, +5 |
| | 7 | −2, +5 |
| 28 | 731 | −724, +7 |
| 29 | 314 | −306, +8 |
| | 319 | −306, +13 |
| | 22 | −7, +15 |
| 31 | 11 | −4, +7 |
| 32 | 23 | −9, +14 |
| | 13 | −6, +7 |
| | 9 | −8, +1 |
| 34 | 6 | −2, +4 |
| 36 | 19 | −14, +5 |
| 38 | 430 | −423, +7 |
| | 28 | −25, +3 |

Interestingly, three small deletions were each found in two founders: a 19 bp deletion in founders 7 and 36, a 21 bp deletion in founders 17 and 27, and a 6 bp deletion in founders 34 and 44 (FIG. 9).

A high rate of germline transmission from Mdr1a founders was observed. Nine of the founders were chosen to backcross to the wild-type FVB/N mice to the F1 generation, all of which transmitted at least one mutant allele to their offspring. Seven founders transmitted multiple mutated alleles. Interestingly, in some cases, novel alleles that were not identified in founders also transmitted germline, such as founders 6, 8, 13, 21, and 44 (Table 2).

TABLE 2
Alleles transmitted in germline

| Founder | Deletion | # | | | % |
|---|---|---|---|---|---|
| ID | (bp) | Hets | Wildtype | Total | Transmission |
| 6 | Small | 5 | 2 | 9 | 77.8 |
| | 694 | 2 | | | |
| 8 | Small | 3 | 0 | 4 | 100.0 |
| | 248 | 1 | | | |
| 11 | 417, 19 | 3 | 3 | 7 | 57.1 |
| | 533 | 1 | | | |
| 13 | 2 | 1 | 0 | 1 | 100.0 |
| 21 | 533 + | 4 | 2 | 12 | 58.3 |
| | 5 bp | | | | |
| | 47 | 1 | | | |
| | 19 | 1 | | | |
| | 21 | 1 | | | |
| 23 | 396 | 14 | 15 | 29 | 48.3 |
| 26 | 534 | 2 | 0 | 15 | 100.0 |
| | 19 | 8 | | | |
| | 11 | 5 | | | |
| 27 | 75 | 4 | 17 | 37 | 54.1 |
| | 19 | 10 | | | |
| | 7 | 6 | | | |
| 44 | 455 | 1 | 6 | 16 | 56.3 |
| | 7 | 1 | | | |
| | 6 | 7 | | | |

To verify that deletion in the Mdr1a gene abolishes its expression, we performed RT-PCR on total RNA from liver, kidney and intestine of Mdr1a−/− mice established from founder 23, with a 396 bp deletion (FIG. 10A), using a forward and a reverse primer located in exons 5 and 9, respectively. The Mdr1a protein is differentially expressed in tissues. Liver and large intestine predominantly express Mdr1a, and kidney expresses both Mdr1a and Mdr1b. Samples from all the Mdr1a−/− tissues produced a smaller product at lower yield than corresponding wild-type samples, with a sequence correlating to exon 7 skipping, which introduces multiple premature stop codons in exon 8 in the mutant animals.

The RT-PCR results demonstrate that the Mdr1a−/− samples produce a transcript missing the 172 bp exon 7 at lower than wild-type level, possibly due to the premature stop codons introduced by exon skipping (FIG. 10B) that lead to non-sense mediated decay. In the Mdr1a−/− samples, there were faint bands at and above the size of the wild-type transcript, which are most likely PCR artifact because amplification of those bands excised from the gel yielded mostly the exon skipped product. The bands at the wild-type size in the second round of PCR were mixtures that did not yield readable sequences (not shown). The mouse Mdr1a gene has 28 exons, and the encoded protein is composed of two units of six transmembrane domains (TMs 1-6 and TMs 7-12) and an ATP binding site with a linker region in between. All 12 TM domains as well as the two ATP-binding motifs are essential for Mdr1a function. The Mdr1a ZFNs target exon 7, which encodes TMs 3 and 4. A partial protein resulting from exon skipping and premature translational terminations will not be functional. The Mdr1a−/− mice derived from founder 23 thus represent a functional knockout.

To validate potential off-target sites of Mdr1a ZFN's, we identified 20 sites in the mouse genome that are most similar to the Mdr1a target site, all with 5 bp mismatches from the ZFN binding sequence. One site is in the Mdr1b gene, which is 88% identical to the Mdr1a gene. To validate the specificity of the Mdr1a ZFNs, we tested the Mdr1b site in all 44 Mdr1a F0 pups using mutation detection assay. None of the 44 pups had an NHEJ event at the Mdr1b site (FIG. 11). The finding that no modifications were detected at the Mdr1b site in any of the 44 live births indicates specificity of the Mdr1a ZFNs. In addition, undesired modifications at loci unlinked to the target site will be lost during subsequent breeding.

Table 3 lists sites among twenty sites in the mouse genome that were checked for off-target activity of Mdr1a ZFNs, which are most similar (with five mismatches) to the Mdr1a target site. Listed are the numbers of the chromosomes they are on and gene names if known. All the mismatched bases are in lower case. The spacer sequence between the binding sites is in bold letters.

TABLE 3
Potential off-target sites for Mdr1a ZFNs

| | | | SEQ |
|---|---|---|---|
| Chr. | Target | | ID |
| No. | Name | Binding Sequence | NO: |
| 5 | Abcb1a | GCCATCAGCCCTGTTCTTGGACTGTCAGCTGGT | 24 |
| 1 | Pld5 | GCCATCAGCtCTCAAAGAGGACTGTaAGaaGcT | 25 |
| 2 | | GCCAaCAGCtCTATTTT-GGACTcTCcGCTGcT | 26 |
| 3 | Slc33a1 | GCCATCAGCtCTATAACAtGACTGTCtaCTGaT | 27 |
| 3 | Syt11 | GtCAcCAaCCCTCTCCATGGAaaGTCAGCTGGT | 28 |
| 4 | | GaCtTCAGCCCTGACTGCtGACTGgCAaCTGGT | 29 |
| 4 | Anp32b | GCCAgCAGCCCTTTCCTTGaAggGTCAGCTaGT | 30 |
| 5 | Pitpnm2 | GCCATCAGCCCgCTCATGaGcCTGTttGCTGGT | 31 |
| 5 | | GCCAgCAGCCCTGCCTG-GGcCTGgCAGtTaGT | 32 |
| 5 | Abcb1b | GCtTCAGCCCTCTTATTGGAtTGTCAtCTGcT | 33 |
| 6 | Mitf | GCCcTCAGCCCTCGAGATGctCTGTCAtCaGGT | 34 |
| 7 | lqck | GCCATCAGCCCaCTGTG-GGACTtTgAGtgGGT | 35 |
| 8 | Kifc3 | caCcTgAGCCCgCAACT-GGACTGTCAGCTGGT | 36 |
| 8 | | cCCATCAaCaCTAACACAGGACTGgCAtCTGGT | 37 |
| 10 | Oprm1 | tCCAgCAGCtCTGTCTG-GGACTGTtAGaTGGT | 38 |
| 10 | Pcbp3 | cCCAaCAGCCCTATTAG-GGACaGgCAcCTGGT | 39 |
| 11 | | GCCATCAGgCaTGGAGA-GGACatTCAGCTGGa | 40 |
| 12 | | GCCATCgcCCCTGGCCT-GGAtgGTCtGCTGGT | 41 |
| 12 | | cCCATCAGCaCTGTGGACGGtCgGTCAtCTGGT | 42 |
| 15 | | GCCAggAGCCtTTCAAGTGGACTGTCAGtTGcT | 43 |
| 16 | EtvS | GCCAgCAGCtgTGACTGTGGgCTaTCAGCTGGT | 44 |

Table 4 below presents the amino acid sequences of helices of the active ZFNs.

TABLE 4
Amino acid sequences of helices of active ZFNs

| | | SEQ |
|---|---|---|
| | | ID |
| Name | Sequence of Zinc Finger Helices | NO: |
| Mdr1a | DRSHLSR TSGNLTR QSSDLSR RSDHLTQ | 45 |
| Mdra | TSGHLSR QSSDLSR QSADRTK RSDVLSE QSGHLSR | 46 |
| Mdr1b | TSGHLSR RSDNLSE RNANRIT RSDHLSE RNDNRKR | 47 |
| Mdr1b | RSDHLSE NNSSRTR TSGHLSR QSSDLRR | 48 |
| MRP1 | TNGQLKE TSSSLSR RSDNLSE ASKTRKN RSDHLTQ | 49 |
| MRP1 | DRSALSR RSDALAR RSDHLSR QSSDLRR RSDVLSE | 50 |
| MRP2 | TSDHLTE DRSNLSR DRSNLTR TSGHLSR QSSDLRR | 51 |
| MRP2 | RSDNLSV QNATRIN RSDALST DRSTRTK RSDDLSR | 52 |
| | RNDNRTK | |
| BCRP | QSGNLAR QSGNLAR RSDSLST DNASRIR DRSNLTR | 53 |
| BCRP | QSSDLSR RNDDRKK RREDLIT TSSNLSR QSGHLSR | 54 |

What is claimed is:

1. A genetically modified animal comprising at least one edited chromosomal sequence involved in ADME and toxicology.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such that no functional protein involved in ADME and toxicology is produced.

4. The genetically modified animal of claim 3, wherein the inactivated chromosomal sequence comprises no exogenously introduced sequence.

5. The genetically modified animal of claim 3, further comprising at least one chromosomally integrated sequence encoding a functional protein involved in ADME and toxicology.

6. The genetically modified animal of 1, wherein the chromosomal sequence encoding the protein involved in ADME and toxicology is chosen from Oct 1, Oct 2, Hfe2, Ppar(alpha), and combinations thereof.

7. The genetically modified animal of claim 1, further comprising a conditional knock-out system for conditional expression of the protein involved in ADME and toxicology.

8. The genetically modified animal of claim 1, wherein the edited chromosomal sequence comprises an integrated reporter sequence.

9. The genetically modified animal of claim 1, wherein the animal is heterozygous or homozygous for the at least one edited chromosomal sequence.

10. The genetically modified animal of claim 1, wherein the animal is an embryo, a juvenile, or an adult.

11. The genetically modified animal of claim 1, wherein the animal is chosen from bovine, canine, equine, feline, ovine, porcine, non-human primate, and rodent.

12. The genetically modified animal of claim 1, wherein the animal is rat.

13. The genetically modified animal of claim 4, wherein the animal is rat and the protein involved in ADME and toxicology is human.

14. A non-human embryo, the embryo comprising at least one RNA molecule encoding a zinc finger nuclease that recognizes a chromosomal sequence involved in ADME and toxicology, and, optionally, at least one donor polynucleotide comprising a sequence encoding an ortholog of a protein involved in ADME and toxicology.

15. The non-human embryo of claim 14, wherein the chromosomal sequence involved in ADME and toxicology is chosen from Oct 1, Oct 2, Hfe2, Ppar(alpha), and combinations thereof.

16. The non-human embryo of claim 14, wherein the embryo is chosen from bovine, canine, equine, feline, ovine, porcine, non-human primate, and rodent.

17. The non-human embryo of claim 114, wherein the embryo is rat and the protein involved in ADME and toxicology is human.

18. A genetically modified cell, the cell comprising at least one edited chromosomal sequence encoding a protein involved in ADME and toxicology.

19. The genetically modified cell of claim 18, wherein the edited chromosomal sequence is inactivated, modified, or comprises an integrated sequence.

20. The genetically modified cell of claim 18, wherein the edited chromosomal sequence is inactivated such that no functional protein involved in ADME and toxicology is produced.

21. The genetically modified cell of claim 20, wherein the inactivated chromosomal sequence comprises no exogenously introduced sequence.

22. The genetically modified cell of claim 21, further comprising at least one chromosomally integrated sequence encoding a functional protein involved in ADME and toxicology.

23. The genetically modified cell of claim 18, wherein the cell is heterozygous or homozygous for the at least one edited chromosomal sequence.

24. The genetically modified cell of claim 18, wherein the cell is of bovine, canine, equine, feline, human, ovine, porcine, non-human primate, or rodent origin.

25. The genetically modified cell of claim 18, wherein the cell is of rat origin and the protein involved in ADME and toxicology is human.

26. The genetically modified cell of claim 18, wherein the chromosomal sequence encoding the protein involved in ADME and toxicology is Oct 1 and at least one of Oct 2, Hfe2, and Ppar(alpha).

27. The genetically modified cell of claim 18, further comprising a conditional knock-out system for conditional expression of the protein involved in ADME and toxicology.

28. The genetically modified cell of claim 18, wherein the edited chromosomal sequence comprises an integrated reporter sequence.

29. A method for assessing the effect of an agent in an animal, the method comprising contacting a genetically modified animal comprising at least one edited chromosomal sequence involved in ADME and toxicology, with an agent, and comparing results of a selected parameter to results obtained from contacting a wild-type animal with the same agent, wherein the selected parameter is chosen from:
a) rate of elimination of the agent or its metabolite(s);
b) circulatory levels of the agent or its metabolite(s);
c) bioavailability of the agent or its metabolite(s);
d) rate of metabolism of the agent or its metabolite(s);
e) rate of clearance of the agent or its metabolite(s);
f) toxicity of the agent or its metabolite(s); and
g) efficacy of the agent or its metabolite(s).

30. The method of claim 29, wherein the agent is a pharmaceutically active ingredient, a drug, a toxin, or a chemical.

31. The method of claim 29, wherein the at least one edited chromosomal sequence is inactivated such that the protein encoded by the chromosomal sequence involved in ADME and toxicology is not produced, and wherein the animal further comprises at least one chromosomally integrated sequence encoding a protein encoded by the chromosomal sequence involved in ADME and toxicology.

32. The method of claim 29, wherein the protein encoded by the chromosomal sequence involved in ADME and toxicology is chosen from Oct 1, Oct 2, Hfe2, Ppar(alpha), and combinations thereof.
